(19) United States
(12) Patent Application Publication
(10) Pub. No.: US 2021/0324398 A1
(43) Pub. Date: Oct. 21, 2021

(54) EDITED NAC GENES IN PLANTS

(71) Applicant: PIONEER HI-BRED INTERNATIONAL, INC., JOHNSTON, IA (US)

(72) Inventors: ZHENGLIN HOU, ANKENY, IA (US); MARY A RUPE, ALTOONA, IA (US); BO SHEN, JOHNSTON, IA (US); ROBERT W WILLIAMS, MINNEAPOLIS, MN (US); WEIQING ZENG, JOHNSTON, IA (US); JUN ZHANG, JOHNSTON, IA (US)

(73) Assignee: PIONEER HI-BRED INTERNATIONAL, INC., JOHNSTON, IA (US)

(21) Appl. No.: 16/972,739

(22) PCT Filed: Jun. 19, 2019
(86) PCT No.: PCT/US19/37991

§ 371 Date: Dec. 7, 2020

Related U.S. Application Data
(60) Provisional application No. 62/692,182, filed on Jun. 29, 2018.

(51) Int. Cl. C12N 15/82

(52) U.S. Cl. CPC C12N 15/8266; C12N 15/8213

(57) ABSTRACT

Compositions and methods for editing an endogenous NAC genes in plants are provided, for the improvement of traits of agronomic or commercial importance. Modifications of expression and activity of NAC genes are described, including editing endogenous NAC gene functional motifs and knocking out NAC gene function.

### CROSS-REFERENCE TO RELATED APPLICATION

This application is a 371 National Stage Entry of PCT Application No. PCT/US19/37991 filed 19 Jun. 2019, which claims the benefit of U.S. Provisional Patent Application Ser. No. 62/692,182 filed 29 Jun. 2018, all of which are herein incorporated by reference in their entireties.

### REFERENCE TO SEQUENCE LISTING SUBMITTED ELECTRONICALLY

The official copy of the sequence listing is submitted electronically via EFS-Web as an ASCII formatted sequence listing with a file named 7765USPCT_SeqListing_ST25.TXT created on 2 Nov. 2020 and having a size of 1,086,390 bytes and is filed concurrently with the specification. The sequence listing comprised in this ASCII formatted document is part of the specification and is herein incorporated by reference in its entirety.

### FIELD OF THE INVENTION

This disclosure relates to compositions and methods of improving traits of agronomic importance in plants.

### BACKGROUND

Yield is a trait of particular economic interest, especially because of increasing world population and the dwindling supply of arable land available for agriculture. Crops such as corn, wheat, rice, canola and soybean account for over half the total human caloric intake, whether through direct consumption of the seeds themselves or through consumption of meat products raised on processed seeds.

Several factors contribute to crop yield. One approach to increase crop yield is to extend the duration of active photosynthesis. The stay-green phenotype has been associated with increases in crop yield. Plants assimilate carbohydrates and nitrogen in vegetative organs (source) and remobilize them to newly developing tissues during development, or to reproductive organs (sink) during senescence. Increasing source strength in cereal crops can lead to increase in grain yield. Staygreen trait (or delayed senescence) during the final stage of leaf development is considered an important trait in increasing source strength in grain production. Staygreen is broadly categorized into two groups, functional and nonfunctional. Functional staygreen is defined as retaining both greenness and photosynthetic competence much longer during senescence.

Recent advances in plant genetic engineering have opened new doors to engineer plants to have improved characteristics or traits. Knockdowns or knockouts of some genes have been demonstrated to provide improved traits of agronomic interest to plants, particularly crop plants. In plants, the NAC genes are a class of transcription factors involved in a variety of functions including embryonic, floral, and vegetative development, lateral root formation and auxin signaling, as well as viral defense and resistance to a variety of biotic and abiotic stresses. Stay-green is valuable trait for improving crop stress tolerance and yield. Delaying leaf senescence may lead to a stay-green phenotype, increased photosynthetic period, and improved source capacity. Novel methods and compositions for delaying leaf senescence in plants are desirable.

### SUMMARY OF INVENTION

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the modification produces an improved trait of agronomic importance.

In any aspect, the modification of an endogenous genomic locus of a plant may be effected by providing to at least one cell of the plant a molecular modification agent. The molecular modification agent may be any molecule known in the art to create a double-strand break or alter the chemical composition of at least one nucleotide in a target sequence. Examples of molecular modification agents include, but are not limited to: Cas endonucleases, zinc finger endonucleases, meganucleases, TAL-Effector nucleases, restriction endonucleases cytidine deaminases, adenine deaminases. In some aspects, the Cas endonuclease forms a functional complex with a guide RNA that comprises a sequence capable of hybridization with a target sequence at or near the endogenous genomic locus.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the modification produces an improved trait of agronomic importance, wherein the trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, stay-green, senescence, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the genetic modification(s) is(are) introduced by an RNA-guided CRISPR endonuclease, a site-specific deaminase, a meganuclease, a zinc-finger nuclease, or a combination thereof.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein insertion or deletion of at least one nucleotide in or near the NAC coding region effects a frameshift in the endogenous NAC gene.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein a regulatory expression element of the endogenous NAC gene is altered.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein at least 1 base, at least 2 bases, at least 3 bases, at least 4 bases, at least 5 bases, or more than 5 bases of the endogenous NAC gene is(are) deleted.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein at least 1 base, at least 2 bases, at least 3 bases, at least 4 bases, at least 5 bases, or more than 5 bases of the endogenous NAC gene is(are) inserted.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein a functional motif of the endogenous NAC gene is replaced or altered.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein a functional motif of the endogenous NAC gene is replaced or altered, wherein the functional motif is selected from the group consisting of: (a) a DNA interaction domain comprising at least two beta sheets and a sequence comprising a tryptophan, an acidic residue, and a basic residue; (b) a protein recognition domain comprising an alpha helix with a plurality of proline residues; (c) a C-terminal domain comprising a tryptophan; (d) an amino acid sequence sharing at least 5 identical amino acids with the sequence YWKATGKDR, wherein one must be tryptophan, one must be an acidic residue, and one must be a basic residue; (e) an amino acid sequence sharing at least 5 identical amino acids with the sequence PATPPPPPLPP, wherein at least 2, at least 3, at least 4, or at least 5 must be proline; and (e) an amino acid sequence sharing at least 5 identical amino acids with the sequence AAGAVVASSAWMNHF, wherein one must be an aromatic residue, in some aspects tryptophan.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein a plurality of sites of the endogenous NAC gene are altered.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein a plurality of sites of the endogenous NAC gene are altered, wherein at least one of the sites is upstream of the coding region.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is a Cas endonuclease.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is a Cas endonuclease lacking nuclease capability, operably linked to a heterologous nuclease.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is a Cas endonuclease lacking nuclease capability, operably linked to a site-specific nuclease.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is a Cas endonuclease further comprising a guide polynucleotide, wherein the guide polynucleotide is substantially complementary to a polynucleotide on, or within 100 nucleotides of, the endogenous NAC gene.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is Cas9 or Cpf1.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein said modifying comprises introducing a double-strand-break-inducing agent to the polynucleotide of the allele, wherein the double-strand-break-inducing agent is a Cas endonuclease further comprising a guide polynucleotide, wherein the guide polynucleotide is substantially complementary to a polynucleotide on, or within 100 nucleotides of, the endogenous NAC gene, wherein two sites are altered with two different guide polynucleotides.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the plant is a monocot.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the plant is maize.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the NAC polypeptide is NAC7.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the NAC polypeptide is NAC7, further comprising introducing a first edit at a position between −291 and −292 bases upstream of the start codon ATG, and introducing a second edit at a position between 122 and 123 bases downstream of the start codon ATG.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the NAC polypeptide is NAC7, wherein the average grain moisture of the kernels from a cob of a plant produced by the method is not more than 1.0%, 1.1%, 1.2%, 1.3%, 1.4%, 1.5%, 1.6%, 1.7%, 1.8%, 1.9%, 2.0%, 3%, 4%, or 5% higher than that of a null control.

In one aspect, the invention provides a method of modifying an endogenous genomic locus of a plant, the locus comprising a polynucleotide encoding a NAC polypeptide, said method comprising introducing one or more genetic modifications comprising polynucleotide insertions, deletions, substitutions, or a combination thereof, such that the genetic modifications result in a reduced expression of the polynucleotide encoding the NAC polypeptide or reduced activity of the NAC polypeptide compared to a control plant not comprising the one or more introduced genetic modifications, wherein the NAC polypeptide is NAC7, wherein the NAC7 polypeptide comprises a sequence that shares at least at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 125, at least 125, between 125 and 150, at least 150, between 150 and 200, at least 200, between 200 and 250, at least 250, between 250 and 300, at least 300, between 300 and 325, and at least 325 contiguous amino acids of any of SEQID NOs: 3, 38-226, or 266-403.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the modification produces an improved trait of agronomic importance.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the modification produces an improved trait of agronomic importance, wherein the trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, stay-green, senescence, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the genetic modification(s) is(are) introduced by an RNA-guided CRISPR endonuclease, a site-specific deaminase, a meganuclease, a zinc-finger nuclease, or a combination thereof.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein insertion or deletion of at least one nucleotide in or near the NAC coding region effects a frameshift in the endogenous NAC gene.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein a regulatory expression element of the endogenous NAC gene is altered.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein at least 1 base, at least 2 bases, at least 3 bases, at least 4 bases, at least 5 bases, or more than 5 bases of the endogenous NAC gene is(are) deleted.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein at least 1 base, at least 2 bases, at least 3 bases, at least 4 bases, at least 5 bases, or more than 5 bases of the endogenous NAC gene is(are) inserted.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein a functional motif of the endogenous NAC gene is replaced or altered.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the functional motif is selected from the group consisting of: (a) a DNA interaction domain comprising at least two beta sheets and a sequence comprising a tryptophan, an acidic residue, and a basic residue; (b) a protein recognition domain comprising an alpha helix with a plurality of proline residues; (c) a C-terminal domain comprising a tryptophan; (d) an amino acid sequence sharing at least 5 identical amino acids with the sequence YWKATGKDR, wherein one must be tryptophan, one must be an acidic residue, and one must be a basic residue; (e) an amino acid sequence sharing at least 5 identical amino acids with the sequence PATPPPPPLPP, wherein at least 2, at least 3, at least 4, or at least 5 must be proline; and (e) an amino acid sequence sharing at least 5 identical amino acids with the sequence AAGAVVASSAWMNHF, wherein one must be an aromatic residue, in some aspects tryptophan.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein a plurality of sites of the endogenous NAC gene are altered.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein a plurality of sites of the endogenous NAC gene are altered, wherein at least one of the sites is upstream of the coding region.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is a Cas endonuclease.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is a Cas endonuclease lacking nuclease capability, operably linked to a heterologous nuclease.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is a Cas endonuclease lacking nuclease capability, operably linked to a site-specific nuclease.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is a Cas endonuclease further comprising a guide polynucleotide, wherein the guide polynucleotide is substantially complementary to a polynucleotide on, or within 100 nucleotides of, the endogenous NAC gene.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is Cas9 or Cpf1.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein said modifying comprises introducing a double-strand-break-inducing agent to the polynucleotide of the allele, wherein the double-strand-break-inducing agent is a Cas endonuclease further comprising a guide polynucleotide, wherein the guide polynucleotide is substantially complementary to a polynucleotide on, or within 100 nucleotides of, the endogenous NAC gene, wherein two sites are altered with two different guide polynucleotides.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the plant is a monocot.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the plant is maize.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the NAC polypeptide is NAC7.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the NAC polypeptide is NAC7, wherein the NAC7 polypeptide comprises a sequence that shares at least at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 125, at least 125, between 125 and 150, at least 150, between 150 and 200, at least 200, between 200 and 250, at least 250, between 250 and 300, at least 300, between 300 and 325, and at least 325 contiguous amino acids of any of SEQID NOs: 3, 38-226, or 266-403.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the NAC polypeptide is NAC7, further comprising introducing a first edit at a position between −291 and −292 bases upstream of the start codon ATG, and introducing a second edit at a position between 122 and 123 bases downstream of the start codon ATG.

In one aspect, the invention provides a method of improving a trait of agronomic importance in a plant, the method comprising introducing, at an endogenous genomic locus of the plant comprising a polynucleotide encoding a NAC polypeptide, one or more genetic modifications polynucleotide insertions, deletions, substitutions or a combination thereof, such that the genetic modifications result in a reduced expression of the polynucleotide encoding the NAC polypeptide or reduced activity of the NAC polypeptide, as compared to a control plant not comprising the edit, wherein the average grain moisture of the kernels from a cob of a plant produced by the method is not more than 2%, 3%, 4%, or 5% higher than that of a null control.

In one aspect, the invention provides a method of altering the binding specificity of a NAC protein in a plant, the method comprising introducing an edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R.

In one aspect, the invention provides a method of altering the binding specificity of a NAC protein in a plant, the method comprising introducing an edit to at least two, at least three, at least four, at least five, at least six, at least seven, at least eight, or nine positions of a contiguous sequence motif N1-N2-N3-N4-N5-N6-N7-N8-N9, wherein the naturally occurring amino acids at each position comprise: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; and N9=K, E, C, G, T, or R.

In one aspect, the invention provides a method of altering the binding specificity of a NAC protein in a plant, the method comprising introducing an edit to at least two, at least three, at least four, at least five, at least six, at least seven, at least eight, or nine positions of a contiguous sequence motif N1-N2-N3-N4-N5-N6-N7-N8-N9, wherein the naturally occurring amino acids at each position consist of: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; and N9=K, E, C, G, T, or R.

In one aspect, the invention provides a method of altering the binding specificity of a NAC protein in a plant, the method comprising introducing an edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the plant is a monocot.

In one aspect, the invention provides a method of altering the binding specificity of a NAC protein in a plant, the method comprising introducing an edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the plant is maize.

In one aspect, the invention provides a method of altering the binding specificity of a NAC protein in a plant, the method comprising introducing an edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the NAC polypeptide is NAC7.

In one aspect, the invention provides a method of altering the binding specificity of a NAC protein in a plant, the method comprising introducing at least two edits to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein a first edit is created at a position between −291 and −292 bases upstream of the start codon ATG, and a second edit is created at a position between 122 and 123 bases downstream of the start codon ATG.

In one aspect, the invention provides a method of altering the binding specificity of a NAC protein in a plant, the method comprising introducing at least two edits to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the NAC protein is NAC7.

In one aspect, the invention provides a method of altering the binding specificity of a NAC protein in a plant, the method comprising introducing at least one edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the NAC7 polypeptide comprises a sequence that shares at least at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 125, at least 125, between 125 and 150, at least 150, between 150 and 200, at least 200, between 200 and 250, at least 250, between 250 and 300, at least 300, between 300 and 325, and at least 325 contiguous amino acids of any of SEQID NOs: 3, 38-226, or 266-403.

In one aspect, the invention provides a method of altering the binding specificity of a NAC protein in a plant, the method comprising introducing at least one edit to a sequence motif comprising: N1-N2-N3-N4-N5-N6-N7-N8-N9, wherein: N1=F, R, T, V, or Y; N2=W; N3=H, K, R, N, or S; N4=S, P, T, I A, or K; N5=T, S, A, V, or E; N6=G, A, or C; N7=R, K, A, S, T, P, or N; N8=D, S, T, E, or P; or N9=K, E, C, G, T, or R; wherein the average grain moisture of the kernels from a cob of a plant produced by the method is not more than 1.0%, 1.1%, 1.2%, 1.3%, 1.4%, 1.5%, 1.6%, 1.7%, 1.8%, 1.9%, or 2.0% higher than that of a null control.

In one aspect, the invention provides a plant comprising at least one modification in at least one allele of a NAC gene, wherein the modification produces an improved trait of agronomic importance in the plant.

In one aspect, the invention provides a plant comprising at least one modification in at least one allele of a NAC gene, wherein the modification produces an improved trait of agronomic importance in the plant, wherein the NAC gene encodes a NAC7 polypeptide.

In one aspect, the invention provides a plant comprising at least one modification in at least one allele of a NAC gene, wherein the modification produces an improved trait of agronomic importance in the plant, wherein the NAC gene encodes a NAC7 polypeptide, wherein the NAC7 polypeptide comprises a sequence that shares at least at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 125, at least 125, between 125 and 150, at least 150, between 150 and 200, at least 200, between 200 and 250, at least 250, between 250 and 300, at least 300, between 300 and 325, and at least 325 contiguous amino acids of any of SEQID NOs: 3, 38-226, or 266-403.

In any aspect, a plant is provided with an edited NAC gene, wherein the plant is a monocot or a dicot.

In any aspect, a plant is provided with an edited NAC gene, wherein the plant is a monocot selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

In any aspect, a plant is provided with an edited NAC gene, wherein the plant is a dicot selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

In any aspect, a plant is provided with an edited NAC gene, wherein the edit imparts an improved trait of agronomic importance to the plant, wherein the trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, stay-green, senescence, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition.

In any aspect, a progeny of a parental plant with an edited NAC gene is provided, wherein the progeny retains the improved trait of agronomic importance imparted to the parental plant, wherein the trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, stay-green, senescence, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition.

In one aspect, a plant produced by any of the methods described herein is provided.

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

FIG. 1 depicts three strategies to generate a non-functional NAC7 allele in maize by genome editing.

FIG. 2 describes the guide RNAs and the targeted editing sites in the NAC7 gene.

FIG. 3 describes sequence changes observed in Event 1 of PHP80319 (CR1: −13 bp; CR2: +1 bp).

FIG. 4 describes sequence changes observed in Event 2 of PHP80319 (CR1: −76 bp; CR2: +1 bp).

FIG. 5 describes sequence changes observed in Event 3 of PHP80319 (CR1: −55 bp; CR2: −5 bp).

FIG. 6 shows knock out of NAC7 delayed dark induced senescence in leaf.

FIG. 7 demonstrates knockdown of NAC7 by the RNAi construct increased percent kernel moisture.

FIG. 8A shows the Zea mays NAC7 N-terminal domain structure predicted from the Arabidopsis analog. The major structure core consists of a 6 stranded beta sheet (β2-β3-β7-β6-β5-β4). FIG. 8B shows alpha helices α2 and α3 flanking the sheet's β2-β3 on both sides while the β5-β4 curled significantly forming semi-barrel with help of β3′. FIG. 8C shows that NAC7 functions as a homodimer, related with a 2-fold axis with its interface formed by the N-terminal peptide including α1-loop-β1.

FIG. 9 shows that the central β-sheet's β4 edge (with the motif YWKATGKDR (SEQID NO:229)) inserts into the DNA duplex major groove, determining the sequence recognition specificity, and shows the major interaction between the NAC7 and its targeting DNA duplex. The specific-determining DNA binding motif residues are labeled along the β4.

FIG. 10 shows the Zea mays NAC7 variant sequence (SEQID NO:3) central β-sheet's β4 edge motif is depicted with a dashed line box, with other elements shown, including loops spanning β3-β3′ loop, β5-β6, and β7-C-ter interact with the DNA phosphate backbone.

FIG. 11 shows conserved regions of the Zea mays NAC7 variant (SEQID NO:3), with the predicted helix areas shown as solid line boxed areas, and the central β-sheet's β4 edge motif depicted with a dashed line box.

FIG. 12A shows a stereo view of aromatic residues interacting with PolyP in a profilin molecule, showing the polyproline (PolyP) segment (PATPPPPPLPP (SEQID NO:230)) that is associated with protein recognition, providing an excellent docking site for aromatic residues. FIG. 12B depicts a three-dimensional model of the PolyP segment.

FIG. 13 shows a phylogenetic tree for the maize NAC proteins, with Glade clustering from the sequences of the central β-sheet's (34 edge region identified.

FIG. 14 shows a multiple sequence alignment of selected Zea mays NAC genes, with the β-sheet's (34 edge region variations outlined in black boxes, with the conserved DNA binding motif highlighted. The secondary structural elements are also labeled such that a is for helix and (3 for beta strand.

SEQID NO:1 is the Genomic DNA of NAC7 in Variety A DNA of Zea mays.

SEQID NO:2 is the CDS of NAC7 in Variety A DNA of Zea mays.

SEQID NO:3 is the Amino acids sequence of NAC7 in Variety A protein of Zea mays.

SEQID NO:4 is the Sequence of Line 1 amplified by GSPs (GSP1+GSP2) DNA of Zea mays.

SEQID NO:5 is the Sequence of Line 1 amplified by GSPs (GSP3+GSP4) DNA of Zea mays.

SEQID NO:6 is the Sequence of Line 2 amplified by GSPs (GSP1+GSP2) DNA of Zea mays.

SEQID NO:7 is the Sequence of Line 2 amplified by GSPs (GSP3+GSP4) DNA of Zea mays.

SEQID NO:8 is the Sequence of Line 3 amplified by GSPs (GSP1+GSP2) DNA of Zea mays.

SEQID NO:9 is the Sequence of Line 3 amplified by GSPs (GSP3+GSP4) DNA of Zea mays.

SEQID NO:10 is the oligo nucleotide forward primer amplifying target NAC7 DNA of Artificial.

SEQID NO:11 is the oligo nucleotide reverse primer amplifying target NAC7 DNA of Artificial.

SEQID NO:12 is the oligo nucleotide forward primer amplifying target NAC7 DNA of Artificial.

SEQID NO:13 is the oligo nucleotide forward primer amplifying target NAC7 DNA of Artificial.

SEQID NO:14 is the Guide RNA targeting CR1 site of NAC7 DNA of Artificial.

SEQID NO:15 is the Guide RNA targeting CR2 site of NAC7 DNA of Artificial.

SEQID NO:16 is the Guide RNA for NAC7 gene deletion as described in Example 8 DNA of Artificial.

SEQID NO:17 is the Guide RNA for NAC7 gene deletion as described in Example 8 DNA of Artificial.

SEQID NO:18 is the Guide RNA for NAC7 gene deletion as described in Example 8 DNA of Artificial.

SEQID NO:19 is the Guide RNA for NAC7 gene deletion as described in Example 8 DNA of Artificial.

SEQID NO:20 is the Guide RNA for DNA binding motif null as described in Example 8 DNA of Artificial.

SEQID NO:21 is the Guide RNA for DNA binding motif null as described in Example 8 DNA of Artificial.

SEQID NO:22 is the Guide RNA for NAC7 promoter editing as described in Example 8 DNA of Artificial.

SEQID NO:23 is the Guide RNA for NAC7 promoter editing as described in Example 8 DNA of Artificial.

SEQID NO:24 is the Guide RNA for NAC7 promoter editing as described in Example 8 DNA of Artificial.

SEQID NO:25 is the Guide RNA for NAC7 promoter editing as described in Example 8 DNA of Artificial.

SEQID NO:26 is the Guide RNA for NAC7 promoter editing as described in Example 8 DNA of Artificial.

SEQID NO:27 is the Guide RNA for NAC7 promoter editing as described in Example 8 DNA of Artificial.

SEQID NO:28 is the Guide RNA for NAC7 promoter editing as described in Example 8 DNA of Artificial.

SEQID NO:29 is the Guide RNA for NAC7 promoter editing as described in Example 8 DNA of Artificial.

SEQID NO:30 is the Cas9 protein protein of Streptococcus pyogenes.

SEQID NO:31 is the AmCYAN1 DNA of Anemonia majano.

SEQID NO:32 is the NPTII DNA of Escherichia coli.

SEQID NO:33 is the ZmODP2 DNA of Zea mays.

SEQID NO:34 is the Kan resistance marker DNA of Escherichia coli.

SEQID NO:35 is the ZM-U6 POLIII CHR8 promoter DNA of Zea mays.

SEQID NO:36 is the ZmWUS2 DNA of Zea mays.

SEQID NO:37 is the cas9 gene DNA of Streptococcus pyogenes.

SEQID NO:38 is the Zea mays NAC gene AC196475.3_FGP005 protein.

SEQID NO:39 is the Zea mays NAC gene AC198937.4_FGP005 protein.

SEQID NO:40 is the Zea mays NAC gene AC203535.4_FGP002 protein.

SEQID NO:41 is the Zea mays NAC gene AC205484.3_FGP005 protein.

SEQID NO:42 is the Zea mays NAC gene AC208663.3_FGP002 protein.

SEQID NO:43 is the Zea mays NAC gene AC211478.3_FGP004 protein.

SEQID NO:44 is the Zea mays NAC gene AC212859.3_FGP008 protein.

SEQID NO:45 is the Zea mays NAC gene AC233865.1_FGP003 protein.

SEQID NO:46 is the Zea mays NAC gene GRMZM2G003715_P01 protein.

SEQID NO:47 is the Zea mays NAC gene GRMZM2G004531_P01 protein.

SEQID NO:48 is the Zea mays NAC gene GRMZM2G008374_P01 protein.

SEQID NO:49 is the Zea mays NAC gene GRMZM2G008374_P02 protein.

SEQID NO:50 is the Zea mays NAC gene GRMZM2G009892_P01 protein.

SEQID NO:51 is the Zea mays NAC gene GRMZM2G009892_P02 protein.

SEQID NO:52 is the Zea mays NAC gene GRMZM2G009892_P03 protein.

SEQID NO:53 is the Zea mays NAC gene GRMZM2G009892_P04 protein.

SEQID NO:54 is the Zea mays NAC gene GRMZM2G011598_P01 protein.

SEQID NO:55 is the Zea mays NAC gene GRMZM2G014653_P01 protein.

SEQID NO:56 is the Zea mays NAC gene GRMZM2G014653_P02 protein.

SEQID NO:57 is the Zea mays NAC gene GRMZM2G014653_P03 protein.

SEQID NO:58 is the Zea mays NAC gene GRMZM2G014653_P04 protein.

SEQID NO:59 is the Zea mays NAC gene GRMZM2G018436_P01 protein.

SEQID NO:60 is the Zea mays NAC gene GRMZM2G018553_P01 protein.

SEQID NO:61 is the Zea mays NAC gene GRMZM2G018553_P02 protein.

SEQID NO:62 is the Zea mays NAC gene GRMZM2G025642_P01 protein.

SEQID NO:63 is the Zea mays NAC gene GRMZM2G025642_P02 protein.

SEQID NO:64 is the Zea mays NAC gene GRMZM2G027309_P01 protein.

SEQID NO:65 is the Zea mays NAC gene GRMZM2G027309_P02 protein.

SEQID NO:66 is the Zea mays NAC gene GRMZM2G030325_P01 protein.

SEQID NO:67 is the Zea mays NAC gene GRMZM2G031001_P01 protein.

SEQID NO:68 is the Zea mays NAC gene GRMZM2G031200_P01 protein.

SEQID NO:69 is the Zea mays NAC gene GRMZM2G031200_P02 protein.

SEQID NO:70 is the Zea mays NAC gene GRMZM2G033014_P01 protein.

SEQID NO:71 is the Zea mays NAC gene GRMZM2G038073_P01 protein.

SEQID NO:72 is the Zea mays NAC gene GRMZM2G041668_P01 protein.

SEQID NO:73 is the Zea mays NAC gene GRMZM2G041746_P01 protein.

SEQID NO:74 is the Zea mays NAC gene GRMZM2G041746_P02 protein.

SEQID NO:75 is the Zea mays NAC gene GRMZM2G042494_P01 protein.

SEQID NO:76 is the Zea mays NAC gene GRMZM2G043813_P01 protein.

SEQID NO:77 is the Zea mays NAC gene GRMZM2G048826_P01 protein.

SEQID NO:78 is the Zea mays NAC gene GRMZM2G052239_P01 protein.

SEQID NO:79 is the Zea mays NAC gene GRMZM2G054252_P01 protein.

SEQID NO:80 is the Zea mays NAC gene GRMZM2G054252_P02 protein.

SEQID NO:81 is the Zea mays NAC gene GRMZM2G054277_P01 protein.

SEQID NO:82 is the Zea mays NAC gene GRMZM2G054277_P02 protein.

SEQID NO:83 is the Zea mays NAC gene GRMZM2G058518_P01 protein.

SEQID NO:84 is the Zea mays NAC gene GRMZM2G059428_P01 protein.

SEQID NO:85 is the Zea mays NAC gene GRMZM2G059428_P02 protein.

SEQID NO:86 is the Zea mays NAC gene GRMZM2G059428_P03 protein.

SEQID NO:87 is the Zea mays NAC gene GRMZM2G060116_P01 protein.

SEQID NO:88 is the Zea mays NAC gene GRMZM2G062009_P01 protein.

SEQID NO:89 is the Zea mays NAC gene GRMZM2G062009_P02 protein.

SEQID NO:90 is the Zea mays NAC gene GRMZM2G062650_P01 protein.

SEQID NO:91 is the Zea mays NAC gene GRMZM2G062650_P02 protein.

SEQID NO:92 is the Zea mays NAC gene GRMZM2G063522_P01 protein.

SEQID NO:93 is the Zea mays NAC gene GRMZM2G064541_P01 protein.

SEQID NO:94 is the Zea mays NAC gene GRMZM2G068973_P01 protein.

SEQID NO:95 is the Zea mays NAC gene GRMZM2G069047_P01 protein.

SEQID NO:96 is the Zea mays NAC gene GRMZM2G069047_P02 protein.

SEQID NO:97 is the Zea mays NAC gene GRMZM2G074358_P01 protein.

SEQID NO:98 is the Zea mays NAC gene GRMZM2G077045_P02 protein.

SEQID NO:99 is the Zea mays NAC gene GRMZM2G078954_P01 protein.

SEQID NO:100 is the Zea mays NAC gene GRMZM2G079632_P01 protein.

SEQID NO:101 is the Zea mays NAC gene GRMZM2G079632_P02 protein.

SEQID NO:102 is the Zea mays NAC gene GRMZM2G081930_P01 protein.

SEQID NO:103 is the Zea mays NAC gene GRMZM2G082709_P01 protein.

SEQID NO:104 is the Zea mays NAC gene GRMZM2G083347_P01 protein.

SEQID NO:105 is the Zea mays NAC gene GRMZM2G083347_P02 protein.

SEQID NO:106 is the Zea mays NAC gene GRMZM2G086768_P01 protein.

SEQID NO:107 is the Zea mays NAC gene GRMZM2G091490_P01 protein.

SEQID NO:108 is the Zea mays NAC gene GRMZM2G092465_P01 protein.

SEQID NO:109 is the Zea mays NAC gene GRMZM2G092465_P03 protein.

SEQID NO:110 is the Zea mays NAC gene GRMZM2G094067_P01 protein.

SEQID NO:111 is the Zea mays NAC gene GRMZM2G099144_P01 protein.

SEQID NO:112 is the Zea mays NAC gene GRMZM2G100583_P01 protein.

SEQID NO:113 is the Zea mays NAC gene GRMZM2G100583_P02 protein.

SEQID NO:114 is the Zea mays NAC gene GRMZM2G100593_P01 protein.

SEQID NO:115 is the Zea mays NAC gene GRMZM2G104074_P01 protein.

SEQID NO:116 is the Zea mays NAC gene GRMZM2G104078_P02 protein.

SEQID NO:117 is the Zea mays NAC gene GRMZM2G104078_P03 protein.

SEQID NO:118 is the Zea mays NAC gene GRMZM2G104400_P01 protein.

SEQID NO:119 is the Zea mays NAC gene GRMZM2G104400_P02 protein.

SEQID NO:120 is the Zea mays NAC gene GRMZM2G109627_P01 protein.

SEQID NO:121 is the Zea mays NAC gene GRMZM2G111770_P01 protein.

SEQID NO:122 is the Zea mays NAC gene GRMZM2G112548_P01 protein.

SEQID NO:123 is the Zea mays NAC gene GRMZM2G112681_P01 protein.

SEQID NO:124 is the Zea mays NAC gene GRMZM2G112681_P02 protein.

SEQID NO:125 is the Zea mays NAC gene GRMZM2G113950_P01 protein.

SEQID NO:126 is the Zea mays NAC gene GRMZM2G114850_P01 protein.

SEQID NO:127 is the Zea mays NAC gene GRMZM2G115721_P01 protein.

SEQID NO:128 is the Zea mays NAC gene GRMZM2G122615_P01 protein.

SEQID NO:129 is the Zea mays NAC gene GRMZM2G123246_P01 protein.

SEQID NO:130 is the Zea mays NAC gene GRMZM2G123667_P02 protein.

SEQID NO:131 is the Zea mays NAC gene GRMZM2G123667_P04 protein.

SEQID NO:132 is the Zea mays NAC gene GRMZM2G123667_P05 protein.

SEQID NO:133 is the Zea mays NAC gene GRMZM2G125777_P01 protein.

SEQID NO:134 is the Zea mays NAC gene GRMZM2G126817_P01 protein.

SEQID NO:135 is the Zea mays NAC gene GRMZM2G126936_P01 protein.

SEQID NO:136 is the Zea mays NAC gene GRMZM2G127379_P01 protein.

SEQID NO:137 is the Zea mays NAC gene GRMZM2G134073_P01 protein.

SEQID NO:138 is the Zea mays NAC gene GRMZM2G134073_P02 protein.

SEQID NO:139 is the Zea mays NAC gene GRMZM2G134687_P01 protein.

SEQID NO:140 is the Zea mays NAC gene GRMZM2G134717_P01 protein.

SEQID NO:141 is the Zea mays NAC gene GRMZM2G139700_P01 protein.

SEQID NO:142 is the Zea mays NAC gene GRMZM2G140901_P01 protein.

SEQID NO:143 is the Zea mays NAC gene GRMZM2G140901_P02 protein.

SEQID NO:144 is the Zea mays NAC gene GRMZM2G146380_P01 protein.

SEQID NO:145 is the Zea mays NAC gene GRMZM2G147867_P01 protein.

SEQID NO:146 is the Zea mays NAC gene GRMZM2G152543_P01 protein.

SEQID NO:147 is the Zea mays NAC gene GRMZM2G154182_P01 protein.

SEQID NO:148 is the Zea mays NAC gene GRMZM2G154182_P02 protein.

SEQID NO:149 is the Zea mays NAC gene GRMZM2G154182_P03 protein.

SEQID NO:150 is the Zea mays NAC gene GRMZM2G155816_P01 protein.

SEQID NO:151 is the Zea mays NAC gene GRMZM2G156977_P01 protein.

SEQID NO:152 is the Zea mays NAC gene GRMZM2G158204_P01 protein.

SEQID NO:153 is the Zea mays NAC gene GRMZM2G159094_P01 protein.

SEQID NO:154 is the Zea mays NAC gene GRMZM2G159500_P01 protein.

SEQID NO:155 is the Zea mays NAC gene GRMZM2G159500_P02 protein.

SEQID NO:156 is the Zea mays NAC gene GRMZM2G162739_P01 protein.

SEQID NO:157 is the Zea mays NAC gene GRMZM2G162739_P02 protein.

SEQID NO:158 is the Zea mays NAC gene GRMZM2G163251_P01 protein.

SEQID NO:159 is the Zea mays NAC gene GRMZM2G163841_P01 protein.

SEQID NO:160 is the Zea mays NAC gene GRMZM2G163843_P01 protein.

SEQID NO:161 is the Zea mays NAC gene GRMZM2G163914_P02 protein.

SEQID NO:162 is the Zea mays NAC gene GRMZM2G163914_P03 protein.

SEQID NO:163 is the Zea mays NAC gene GRMZM2G166721_P01 protein.

SEQID NO:164 is the Zea mays NAC gene GRMZM2G166721_P02 protein.

SEQID NO:165 is the Zea mays NAC gene GRMZM2G166721_P03 protein.

SEQID NO:166 is the Zea mays NAC gene GRMZM2G167018_P01 protein.

SEQID NO:167 is the Zea mays NAC gene GRMZM2G167492_P01 protein.

SEQID NO:168 is the Zea mays NAC gene GRMZM2G171395_P01 protein.

SEQID NO:169 is the Zea mays NAC gene GRMZM2G172264_P01 protein.

SEQID NO:170 is the Zea mays NAC gene GRMZM2G174070_P01 protein.

SEQID NO:171 is the Zea mays NAC gene GRMZM2G176677_P01 protein.

SEQID NO:172 is the Zea mays NAC gene GRMZM2G176677_P02 protein.

SEQID NO:173 is the Zea mays NAC gene GRMZM2G176677_P04 protein.

SEQID NO:174 is the Zea mays NAC gene GRMZM2G178998_P01 protein.

SEQID NO:175 is the Zea mays NAC gene GRMZM2G178998_P02 protein.

SEQID NO:176 is the Zea mays NAC gene GRMZM2G179049_P01 protein.

SEQID NO:177 is the Zea mays NAC gene GRMZM2G179049_P02 protein.

SEQID NO:178 is the Zea mays NAC gene GRMZM2G179885_P01 protein.

SEQID NO:179 is the Zea mays NAC gene GRMZM2G179885_P02 protein.

SEQID NO:180 is the Zea mays NAC gene GRMZM2G179885_P03 protein.

SEQID NO:181 is the Zea mays NAC gene GRMZM2G179885_P04 protein.

SEQID NO:182 is the Zea mays NAC gene GRMZM2G180328_P01 protein.

SEQID NO:183 is the Zea mays NAC gene GRMZM2G181605_P01 protein.

SEQID NO:184 is the Zea mays NAC gene GRMZM2G312201_P01 protein.

SEQID NO:185 is the Zea mays NAC gene GRMZM2G312201_P02 protein.

SEQID NO:186 is the Zea mays NAC gene GRMZM2G312201_P03 protein.

SEQID NO:187 is the Zea mays NAC gene GRMZM2G312201_P04 protein.

SEQID NO:188 is the Zea mays NAC gene GRMZM2G315140_P01 protein.

SEQID NO:189 is the Zea mays NAC gene GRMZM2G316840_P01 protein.

SEQID NO:190 is the Zea mays NAC gene GRMZM2G336533_P01 protein.

SEQID NO:191 is the Zea mays NAC gene GRMZM2G336533_P02 protein.

SEQID NO:192 is the Zea mays NAC gene GRMZM2G342647_P01 protein.

SEQID NO:193 is the Zea mays NAC gene GRMZM2G347043_P01 protein.

SEQID NO:194 is the Zea mays NAC gene GRMZM2G354151_P01 protein.

SEQID NO:195 is the Zea mays NAC gene GRMZM2G379608_P01 protein.

SEQID NO:196 is the Zea mays NAC gene GRMZM2G386163_P01 protein.

SEQID NO:197 is the Zea mays NAC gene GRMZM2G386163_P02 protein.

SEQID NO:198 is the Zea mays NAC gene GRMZM2G389557_P01 protein.

SEQID NO:199 is the Zea mays NAC gene GRMZM2G393433_P01 protein.

SEQID NO:200 is the Zea mays NAC gene GRMZM2G393433_P02 protein.

SEQID NO:201 is the Zea mays NAC gene GRMZM2G406204_P01 protein.

SEQID NO:202 is the Zea mays NAC gene GRMZM2G430522_P01 protein.

SEQID NO:203 is the Zea mays NAC gene GRMZM2G430522_P02 protein.

SEQID NO:204 is the Zea mays NAC gene GRMZM2G430522_P03 protein.

SEQID NO:205 is the Zea mays NAC gene GRMZM2G430849_P01 protein.

SEQID NO:206 is the Zea mays NAC gene GRMZM2G435824_P01 protein.

SEQID NO:207 is the Zea mays NAC gene GRMZM2G439903_P01 protein.

SEQID NO:208 is the Zea mays NAC gene GRMZM2G440219_P01 protein.

SEQID NO:209 is the Zea mays NAC gene GRMZM2G450445_P01 protein.

SEQID NO:210 is the Zea mays NAC gene GRMZM2G450445_P02 protein.

SEQID NO:211 is the Zea mays NAC gene GRMZM2G456568_P01 protein.

SEQID NO:212 is the Zea mays NAC gene GRMZM2G456568_P02 protein.

SEQID NO:213 is the Zea mays NAC gene GRMZM2G459156_P01 protein.

SEQID NO:214 is the Zea mays NAC gene GRMZM2G465835_P01 protein.

SEQID NO:215 is the Zea mays NAC gene GRMZM2G475014_P01 protein.

SEQID NO:216 is the Zea mays NAC gene GRMZM2G479980_P01 protein.

SEQID NO:217 is the Zea mays NAC gene GRMZM5G803888_P01 protein.

SEQID NO:218 is the Zea mays NAC gene GRMZM5G813651_P01 protein.

SEQID NO:219 is the Zea mays NAC gene GRMZM5G813651_P02 protein.

SEQID NO:220 is the Zea mays NAC gene GRMZM5G832473_P01 protein.

SEQID NO:221 is the Zea mays NAC gene GRMZM5G857701_P01 protein.

SEQID NO:222 is the Zea mays NAC gene GRMZM5G885329_P01 protein.

SEQID NO:223 is the Zea mays NAC gene GRMZM5G894234_P01 protein.

SEQID NO:224 is the Zea mays NAC gene GRMZM5G898290_P01 protein.

SEQID NO:225 is the Zea mays NAC gene GRMZM5G898290_P02 protein.

SEQID NO:226 is the Zea mays NAC gene GRMZM6G257110_P01 protein.

SEQID NO:227 is the Arabidopsis NAC domain-containing protein 19 3SWM protein of Arabidopsis thaliana.

SEQID NO:228 is the Rice Stress-induced transcription factor NAC1 protein of Oryza sativa.

SEQID NO:229 is the central β-sheet's (34 edge motif protein of Zea mays.

SEQID NO:230 is the polyproline segment associated with protein recognition protein of Zea mays.

SEQID NO:231 is the C-terminal motif protein of Zea mays.

SEQID NO:232 is the central β-sheet's (34 edge motif protein of Zea mays.

SEQID NO:233 is the central β-sheet's (34 edge motif protein of Zea mays.

SEQID NO:234 is the central β-sheet's (34 edge motif protein of Zea mays.

SEQID NO:235 is the replacement for DNA binding motif protein of Artificial.

SEQID NO:236 is the target sequence ZM-NAC7-CR1 DNA of Zea mays.

SEQID NO:237 is the target sequence ZM-NAC7-CR2 DNA of Zea mays.

SEQID NO:238 is an exemplary NAC protein motif variation in maize protein of Zea mays.

SEQID NO:239 is an exemplary NAC protein motif variation in maize protein of Zea mays.

SEQID NO:240 is an exemplary NAC protein motif variation in maize protein of Zea mays.

SEQID NO:241 is an exemplary NAC protein motif variation in maize protein of Zea mays.

SEQID NO:242 is an exemplary NAC protein motif variation in maize protein of Zea mays.

SEQID NO:243 is an exemplary NAC protein motif variation in maize protein of Zea mays.

SEQID NO:244 is an exemplary NAC protein motif variation in maize protein of Zea mays.

SEQID NO:245 is an exemplary NAC protein motif variation in maize protein of Zea mays.

SEQID NO:246 is an exemplary NAC protein motif variation in maize protein of Zea mays.

SEQID NO:247 is an exemplary NAC protein motif variation in maize protein of Zea mays.

SEQID NO:248 is an exemplary NAC protein motif variation in maize protein of Zea mays.

SEQID NO:249 is an exemplary NAC protein motif variation in maize protein of Zea mays.

SEQID NO:250 is an exemplary NAC protein motif variation in maize protein of Zea mays.

SEQID NO:251 is an exemplary NAC protein motif variation in maize protein of Zea mays.

SEQID NO:252 is an exemplary NAC protein motif variation in maize protein of Zea mays.

SEQID NO:253 is an exemplary NAC protein motif variation in maize protein of Zea mays.

SEQID NO:254 is an exemplary NAC protein motif variation in maize protein of Zea mays.

SEQID NO:255 is an exemplary NAC protein motif variation in maize protein of Zea mays.

SEQID NO:256 is an exemplary NAC protein motif variation in maize protein of Zea mays.

SEQID NO:257 is an exemplary NAC protein motif variation in maize protein of Zea mays.

SEQID NO:258 is an exemplary NAC protein motif variation in maize protein of Zea mays.

SEQID NO:259 is an exemplary NAC protein motif variation in maize protein of Zea mays.

SEQID NO:260 is an exemplary NAC protein motif variation in maize protein of Zea mays.

SEQID NO:261 is an exemplary NAC protein motif variation in maize protein of Zea mays.

SEQID NO:262 is an exemplary NAC protein motif variation in maize protein of Zea mays.

SEQID NO:263 is an exemplary NAC protein motif variation in maize protein of Zea mays.

SEQID NO:264 is an exemplary NAC protein motif variation in maize protein of Zea mays.

SEQID NO:265 is an exemplary NAC protein motif variation in maize protein of Zea mays.

SEQID NOs: 266-403 are sequences of NAC proteins.

### DETAILED DESCRIPTION

Various compositions and methods for decreasing expression of the NAC gene in a cell, for example a plant cell, via gene editing are provided. In some aspects, the NAC gene is NAC7.

Terms used in the claims and specification are defined as set forth below unless otherwise specified. It must be noted that, as used in the specification and the appended claims, the singular forms “a,” “an” and “the” include plural referents unless the context clearly dictates otherwise.

### Definitions

The terms “provided (to)” and “introduced (into)” are used interchangeably herein. In another aspect, it is meant that a particular composition becomes functionally associated with a cell or other molecule. In one aspect, it is meant that a particular composition is taken up by the cell into its interior. “Introducing” is intended to mean presenting to a target, such as a cell or organism, a polynucleotide or polypeptide or polynucleotide-protein complex, in such a manner that the component(s) gains access to the interior of a cell of the organism or to the cell itself.

By the term “endogenous” it is meant a sequence or other molecule that naturally occurs in a cell or organism. In one aspect, an endogenous polynucleotide is normally found in the genome of a cell; that is, not heterologous.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more compositions, such as those provided herein, may be entirely synthetic.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The relationship between two or more polynucleotides or polypeptides may be determined. Polynucleotide and polypeptide sequences, fragments thereof, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment.

Sequence relationships may be defined by their composition comparisons, or by their ability to hybridize, or by their ability to engage in homologous recombination.

Methods of alignment of sequences for comparison are well known in the art. Thus, the determination of percent sequence identity between any two sequences can be accomplished using a mathematical algorithm. Sequence alignments and percent identity or similarity calculations may be determined using a variety of comparison methods designed to detect homologous sequences including, but not limited to, the MegAlign program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

“Sequence identity” or “identity” in the context of nucleic acid or polypeptide sequences refers to the nucleic acid bases or amino acid residues in two sequences that are the same when aligned for maximum correspondence over a specified comparison window. The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=S and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program. Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in lx to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, an “isolated” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture media components when produced by recombinant techniques, or substantially free of chemical precursors or other molecules when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the invention or biologically active portion thereof is recombinantly produced, optimally culture medium represents less than about 30%, 20%, 10%, 5%, or 1% (by dry weight) of chemical precursors or non-protein-of-interest molecules.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of another organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene, is recombinant.

The terms “recombinant polynucleotide”, “recombinant nucleotide”, “recombinant DNA” and “recombinant DNA construct” are used interchangeably herein. A recombinant construct comprises an artificial or heterologous combination of nucleic acid sequences, e.g., regulatory and coding sequences that are not found together in nature. For example, a transfer cassette can comprise restriction sites and a heterologous polynucleotide of interest. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments provided herein. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain events displaying the desired expression level and pattern. Such screening may be accomplished by Southern analysis of DNA, Northern analysis of mRNA expression, immunoblotting analysis of protein expression, or phenotypic analysis, among others.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

“Open reading frame” is abbreviated ORF.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225 236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated yet has an effect on cellular processes. The terms “complement” and “reverse complement” are used interchangeably herein with respect to mRNA transcripts, and are meant to define the antisense RNA of the message.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product (e.g., an mRNA, guide RNA, or a protein) in either precursor or mature form.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

The term “fragment” refers to a contiguous set of polynucleotides or polypeptides. In one embodiment, a fragment is 2, 3, 4, 5, 6, 7 8, 9, 10, 11, 12, 13, 14, 15, 16, 17, 18, 19, 20, or greater than 20 contiguous polynucleotides. In one embodiment, a fragment is 2, 3, 4, 5, 6, 7 8, 9, 10, 11, 12, 13, 14, 15, 16, 17, 18, 19, 20, or greater than 20 contiguous polypeptides. A fragment may or may not exhibit the function of a sequence sharing some percent identity over the length of said fragment.

The terms “fragment that is functionally equivalent”, “functional fragment”, and “functionally equivalent fragment” are used interchangeably herein. These terms refer to a portion or subsequence of a nucleic acid fragment or polypeptide that displays the same activity or function as the longer sequence from which it derives. In one example, the fragment retains the ability to alter gene expression, create a double strand nick or break, or produce a certain phenotype whether or not the fragment encodes the whole protein as found in nature. In some aspects, part of the activity is retained. In some aspects, all of the activity is retained.

The terms “variant that is functionally equivalent”, “functional variant”, and “functionally equivalent variant” are used interchangeably herein. These terms refer to a nucleic acid fragment or polypeptide that displays the same activity or function as the source sequence from which it derives, but differs from the source sequence by at least one nucleotide or amino acid. In one example, the variant retains the ability to alter gene expression, create a double strand nick or break, or produce a certain phenotype. In some aspects, part of the activity is retained. In some aspects, all of the activity is retained.

A functional fragment or functional variant shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native source polynucleotide or polypeptide, and retains at least partial activity.

“Modified”, “edited”, or “altered, with respect to a polynucleotide or target sequence, refers to a nucleotide sequence that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) association of another molecule or atom via covalent, ionic, or hydrogen bonding, or (v) any combination of (i)-(iv).

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

An “optimized” polynucleotide is a sequence that has been optimized for improved expression in a particular heterologous host cell.

A “codon-modified gene” or “codon-preferred gene” or “codon-optimized gene” is a gene having its frequency of codon usage designed to mimic the frequency of preferred codon usage of the host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gown (1990) Plant Physiol. 92:1-11 for a discussion of host-preferred codon usage.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

A “polynucleotide of interest” includes any nucleotide sequence encoding a protein or polypeptide that improves desirability of an organism, for example, animals or plants. Polynucleotides of interest: include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

As used herein, a “genomic region of interest” is a segment of a chromosome in the genome of a plant that is desirable for introducing a double-strand break, a polynucleotide of interest, or a trait of interest. The genomic region of interest can include, for example, one or more polynucleotides of interest. Generally, a genomic region of interest of the present invention comprises a segment of chromosome that is 0-15 centimorgan (cM).

The terms “knock-out”, “gene knock-out” and “genetic knock-out” are used interchangeably herein. A knock-out represents a DNA sequence of a cell that has been rendered partially or completely inoperative by targeting with a DSB agent; for example, a DNA sequence prior to knock-out could have encoded an amino acid sequence, or could have had a regulatory function (e.g., promoter).

The terms “knock-in”, “gene knock-in”, “gene insertion” and “genetic knock-in” are used interchangeably herein. A knock-in represents the replacement or insertion of a DNA sequence at a specific DNA sequence in cell by targeting with a DSB agent (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

As used herein, the terms “target site”, “target sequence”, and “target polynucleotide” are used interchangeably herein and refer to a polynucleotide sequence in the genome of a plant cell or yeast cell that comprises a recognition site for a double-strand-break-inducing agent.

A “target cell” is a cell that comprises a target sequence and is the object for receipt of a particular double-strand-break-inducing agent.

A “break-inducing agent” is a composition that creates a cleavage in at least one strand of a polynucleotide. In some aspect, a break-inducing agent may be capable of, or have its activity altered such that it is capable of, creating a break in only one strand of a polynucleotide. Producing a single-strand-break in a double-stranded target sequence may be referred to herein as “nicking” the target sequence.

The term “double-strand-break-inducing agent”, or equivalently “double-strand-break agent” or “DSB agent”, as used herein refers to any composition which produces a double-strand break in a target polynucleotide sequence; that is, creates a break in both strands of a double stranded polynucleotide. Examples of a DSB agent include, but are not limited to: meganucleases, TAL effector nucleases, Argonautes, Zinc Finger nucleases, and Cas endonucleases (either individually or as part of a ribonucleoprotein complex). Producing the double-strand break in a target sequence may be referred to herein as “cutting” or “cleaving” the target sequence. In some aspects, the DSB agent is a nuclease. In some aspects, the DSB agent is an endonuclease. An “endonuclease” refers to an enzyme that cleaves the phosphodiester bond within a polynucleotide chain. In some embodiments, the double-strand break results in a “blunt” end of a double-stranded polynucleotide, wherein both strands are cut directly across from each other with no nucleotide overhang generated. A “blunt” end cut of a double-stranded polynucleotide is created when a first cleavage of the first stand polynucleotide backbone occurs between a first set of two nucleotides on one strand, and a second cleavage of the second strand polynucleotide backbone occurs between a second set of two nucleotides on the opposite strand, wherein each of the two nucleotides of the first set are hydrogen bonded to one of the two nucleotides of the second set, resulting in cut strands with no nucleotide on the cleaved end that is not hydrogen bonded to another nucleotide on the opposite strand. In some embodiments, the double-strand break results in a “sticky” end of a double-stranded polynucleotide, wherein cuts are made between nucleotides of dissimilar relative positions on each of the two strands, resulting in a polynucleotide overhang of one strand compared to the other. A “sticky” end cut of a double-stranded polynucleotide is created when a first cleavage of the first strand polynucleotide backbone occurs between a first set of two nucleotides on one strand, and a second cleavage of the second strand polynucleotide backbone occurs between a second set of two nucleotides on the opposite strand, wherein no more than one nucleotide of the first set is hydrogen bonded to one of the nucleotides of the second set on the opposite strand, resulting in an “overhang” of at least one polynucleotide on one of the two strands wherein the lengths of the two resulting cut strands are not identical. In some embodiments, the DSB agent comprises more than one type of molecule. In one non-limiting example, the DSB agent comprises an endonuclease protein and a polynucleotide, for example a Cas endonuclease and a guide RNA. In some aspects, the DSB agent is a fusion protein comprising a plurality of polypeptides. In one non-limiting example, the DSB agent is a Cas endonuclease with a deactivated nuclease domain, and another polypeptide with nuclease activity.

As used herein, the term “recognition site” refers to a polynucleotide sequence to which a double-strand-break-inducing agent is capable of alignment, and may optionally contact, bind, and/or effect a double-strand break. The terms “recognition site” and “recognition sequence” are used interchangeably herein. The recognition site can be an endogenous site in a host (such as a yeast, animal, or plant) genome, or alternatively, the recognition site can be heterologous to the host (yeast, animal, or plant) and thereby not be naturally occurring in the genome, or the recognition site can be found in a heterologous genomic location compared to where it occurs in nature. The length and the composition of a recognition site can be characteristic of, and may be specific to, a particular double-strand-break-inducing agent. The cleavage site of a DSB agent may be the same or different than the recognition site, and may be the same or different than the binding site.

As used herein, the term “endogenous recognition (or binding or cleavage) site” refers to a double-strand-break-inducing agent recognition (or binding or cleavage) site that is endogenous or native to the genome of a host (such as a plant, animal, or yeast) and is located at the endogenous or native position of that recognition (or binding or cleavage) site in the genome of the host (such as a plant, animal, or yeast). The length of the recognition (or binding or cleavage) site can vary, and includes, for example, recognition (or binding or cleavage) sites that are at least 1, 2, 3, 4, 5, 6, 7, 8, 9, 10, 11, 12, 13, 14, 15, 16, 17. 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more than 70 nucleotides in length. The composition of the recognition (or binding or cleavage) site can vary, and includes, for example, a plurality of specific nucleotides whose compositions are recognized by the DSB agent. In some aspects, the plurality of specific nucleotides is contiguous in the primary sequence. In some aspects, the plurality of specific nucleotides is non-contiguous in the primary sequence. It is further possible that the recognition site could be palindromic, that is, the sequence on one strand reads the same in the opposite direction on the complementary strand. The binding and/or nick/cleavage site could be within the recognition sequence or the binding and/or nick/cleavage site could be outside of the recognition sequence. In another variation, the DSB cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

As used herein, the term “target recognition site” refers to the polynucleotide sequence to which a double-strand-break-inducing agent is capable of aligning perfectly (i.e., zero nucleotide mismatches, gaps, or insertions), and in some aspects, induces a double-strand break.

As used herein, the term “target binding site” refers to the polynucleotide sequence at which the double-strand-break-inducing agent is capable of forming a functional association, and to which it forms bonds with complementary nucleotides of the target polynucleotide strand, with perfect alignment (i.e., zero nucleotide mismatches, gaps, or insertions).

As used herein, the term “target cleavage site” refers to the polynucleotide sequence at which a double-strand-break-inducing agent is capable of producing a double-strand break, with perfect alignment (i.e., zero nucleotide mismatches, gaps, or insertions).

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

The term “Cas protein” refers to a polypeptide encoded by a Cas (CRISPR-associated) gene. A Cas protein includes but is not limited to: the novel Cas-delta protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The Cas-delta endonucleases of the disclosure may include those having RuvC or RuvC-like nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of an organism, produced via sexual or asexual reproduction.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, stay-green, senescence, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

“Stay-green” or “staygreen” is a term used to describe a plant phenotype, e.g., whereby leaf senescence (most easily distinguished by yellowing of leaf associated with chlorophyll degradation) is delayed compared to a standard reference or a control. The staygreen phenotype has been used as selective criterion for the development of improved varieties of crop plants such as corn, rice and sorghum, particularly with regard to the development of stress tolerance, and yield enhancement (Borrell et al. (2000b) Crop Sci. 40:1037-1048; Spano et al, (2003) J. Exp. Bot. 54:1415-1420; Christopher et al, (2008) Aust. J. Agric. Res. 59:354-364, 2008, Kashiwagi et al (2006) Plant Physiology and Biochemistry 44:152-157, 2006 and Zheng et al, (2009) Plant Breed 725:54-62.

“Increase in staygreen phenotype” as referred to in here, indicates retention of green leaves, delayed foliar senescence and significantly healthier canopy in a plant, compared to control plant.

Staygreen plants have been categorized broadly into “cosmetic staygreen” and “functional staygreen”. In plants exhibiting cosmetic staygreen phenotype, the primary lesion of senescence is confined to pigment catabolism. In plants exhibiting functional staygreen phenotype the entire senescence syndrome, of which chlorophyll catabolism is only one component, is delayed or slowed down, or both. The functional staygreen trait has been shown to be associated with the transition from the carbon (C) capture to the nitrogen (N) mobilization phase of foliar development (Thomas and Oughan (2014) J Exp Bot. Vol. 65 (14), pp. 3889-3900; Kusaba et al (2013) Photosynth Res 117:221-234; Thomas and Howarth (2000) J Exp Bot. Vol. 51, pp. 329-337.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%), at least about 400% or more higher than the control.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### NAC Transcription Factors

NAC (Petunia NAM, Arabidopsis ATAF1/2 and CUC2) proteins belong to a plant-specific transcription factor superfamily, whose members comprise a conserved sequence known as the DNA-binding NAC-domain in the N-terminal region and a variable transcriptional regulatory C-terminal region. Based on its motif distribution, the NAC-domain can be divided into five sub-domains (A-E) (Zhu et al Evolution 66-6: 1833-1848; Ooka et al. (2003) DNA Research 10,239-247). The C-terminal regions of some NAC TFs (transcription factors) also contain transmembrane motifs (TMs), which anchor to the plasma membrane. (Lu et al (2012) Plant Cell Rep 31:1701-1711; Tran et al. (2004) Plant Cell 16:2481-2498). At least 117 and 151 NAC family members have been predicted in Arabidopsis and rice, respectively (Nuruzzaman et al. (2010) Gene 465:30-44).

NAC proteins have been implicated in several important pathways, including senescence initiation, such as the Arabidopsis NAC transcription factor, AtNAP, and the GPC protein in wheat (Uauy et al (2006) Science, 24 November, vol 314; Thomas and Ougham Journal of Experimental Botany, Vol. 65, No. 14, pp. 3889-3900, 2014; Lee et al Plant J. (2012) 70, 831-844; Guo and Gan (2006) Plant J. 46,601-612.

Overexpression of some NAC family proteins, such as JUB1 in Arabidopsis thaliana has been shown to strongly delay senescence and enhance tolerance to various abiotic stresses (Wu et al (2012) Plant Cell, Vol. 24: 482-506. Overexpression of some NAC genes has been shown to significantly increase the drought and salt tolerance of a number of plants (Zheng et al. (2009) Biochem. Biophys. Res. Commun. 379:985-989; Lu et al (2012) Plant Cell Rep 31:1701-1711). Transgenic Arabidopsis plants overexpressing ZmSNAC1, a Zea mays NAC1 have been shown to exhibit enhanced sensitivity to ABA and osmotic stress in the germination stage, and exhibited increased tolerance to dehydration in the seedling stage. (Lu et al Plant Cell Rep (2012) 31:1701-1711).

NAC proteins have also been implicated in transcriptional control in a variety of plant processes, including in the development of the shoot apical meristem and floral organs, and in the formation of lateral roots. Arabidopsis NAC gene CUC3 has been reported to contribute to the establishment of the cotyledon boundary and the shoot meristem (Li et al. (2012) BMC Plant Biology, 12:220).

NAC proteins have also been implicated in responses to stress and viral infections (Ernst et al. (2004), EMBO Reports 5, 3, 297-303; Guo and Gan Plant Journal (2006) 46, 601-612, Yoon et al. Mol. Cells, Vol. 25, No. 3, pp. 438-445).

In some embodiments, a NAC protein includes a polypeptide selected from the group consisting of SEQID NOs: AAA-BBB. % ID, etc.

In some embodiments, a NAC protein is encoded by a polynucleotide selected from the group consisting of SEQID NOs: CCC-DDD. % ID, etc.

### Gene Editing

Methods to modify or alter endogenous genomic DNA are known in the art. In some aspects, methods and compositions are provided for modifying naturally-occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks, and includes homologous recombination (HR) and single-strand annealing (SSA) (Lieber. 2010 Annu. Rev. Biochem. 79:181-211).

In addition to the double-strand break inducing agents, site-specific base conversions can also be achieved to engineer one or more nucleotide changes to create one or more edits described herein into the genome. These include for example, a site-specific base edit mediated by a C⋅G to T⋅A or an A⋅T to G⋅C base editing deaminase enzymes (Gaudelli et al., Programmable base editing of A⋅T to G⋅C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. Catalytically dead dCas9 fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA.

### CRISPR-Cas Systems for Gene Editing

CRISPR (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

Endonucleases identified from CRISPR systems may be used to edit a particular polynucleotide, in vitro or in vivo, to effect changes such as nucleotide substitutions, deletions, insertions, or any combination thereof.

### Cas Endonucleases

As used herein, the term “Cas gene” refers to a gene that is generally coupled, associated or close to or in the vicinity of flanking CRISPR loci.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position.

Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases include, but are not limited to, Cas endonucleases identified from the following systems: Class 1, Class 2, Type I, Type II, Type III, Type IV, Type V, and Type VI. In some aspects, the Cas endonuclease is Cas3 (a feature of Class 1 type I systems), Cas9 (a feature of Class 2 type II systems) or Cas12 (Cpf1) (a feature of Class 2 type V systems).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10: 957-963).

A Cas endonuclease, effector protein, functional variant, or a functional fragment thereof, for use in the disclosed methods, can be isolated from a native source, or from a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

To facilitate optimal expression and nuclear localization for eukaryotic cells, the gene comprising the Cas endonuclease may be optimized as described in WO2016186953 published 24 Nov. 2016, and then delivered into cells as DNA expression cassettes by methods known in the art. In some aspects, the Cas endonuclease is provided as a polypeptide. In some aspects, the Cas endonuclease is provided as a polynucleotide encoding a polypeptide. In some aspects, the guide RNA is provided as a DNA molecule encoding one or more RNA molecules. In some aspects, the guide RNA is provide as RNA or chemically-modified RNA. In some aspects, the Cas endonuclease protein and guide RNA are provided as a ribonucleoprotein complex (RNP).

### Guide RNAs

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with respect to each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences.

The tracrRNA (trans-activating CRISPR RNA) comprises, in the 5′-to-3′ direction, (i) an “anti-repeat” sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015). In some embodiments, the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides.

In one embodiment, the RNA that guides the RNA/Cas endonuclease complex is a duplexed RNA comprising a duplex crRNA-tracrRNA.

The guide RNA includes a dual molecule comprising a chimeric non-naturally occurring crRNA linked to at least one tracrRNA. A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limited to Xie et al. 2015, PNAS 112:3570-3575).

In one aspect, the functional variant of the guide RNA can form a guide RNA/Cas9 endonuclease complex that can recognize, bind to, and optionally nick or cleave a target sequence.

Cas endonucleases may be capable of forming a complex with a guide polynucleotide (e.g., guide RNA or gRNA) that is capable of recognizing, binding to, and optionally nicking, unwinding, or cleaving all or part of a target sequence. In some aspects, the guide polynucleotide/Cas endonuclease complex is capable of introducing a double-strand-break into a target polynucleotide. In some aspects, the guide polynucleotide comprises solely RNA, solely DNA, a chimeric molecule comprising both DNA and RNA, and/or comprises a chemically modified nucleotide. The guide polynucleotide (e.g., guide RNA) may be a single guide RNA (sgRNA) that is capable of binding to a sequence on the target polynucleotide.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. In some aspects, the Cas endonuclease may not successfully recognize a target DNA sequence if the target DNA sequence is not adjacent to, or near, a PAM sequence. In some aspects, the PAM precedes the target sequence (e.g. Cas12a). In some aspects, the PAM follows the target sequence (e.g. S. pyogenes Cas9). The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position. It is understood that based on the methods and embodiments described herein utilizing a novel guided Cas system one skilled in the art can now tailor these methods such that they can utilize any guided endonuclease system.

### Cas9-gRNA Mediated Gene Editing

The process for editing a genomic sequence at a Cas9-gRNA double-strand-break site with a modification template generally comprises: providing a host cell with a Cas9-gRNA complex that recognizes a target sequence in the genome of the host cell and is able to induce a double-strand-break in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the double-strand break. Genome editing using double-strand-break-inducing agents, such as Cas9-gRNA complexes, has been described, for example in US20150082478 published on 19 Mar. 2015, WO2015026886 published on 26 Feb. 2015, WO2016007347 published 14 Jan. 2016, and WO2016025131 published on 18 Feb. 2016.

A guide polynucleotide/Cas endonuclease complex described herein is capable of recognizing, binding to, and optionally nicking, unwinding, or cleaving all or part of a target sequence.

Some uses for guide RNA/Cas9 endonuclease systems include but are not limited to modifying or replacing nucleotide sequences of interest (such as a regulatory elements), insertion of polynucleotides of interest, gene knock-out, gene-knock in, gene knock-down, modification of splicing sites and/or introducing alternate splicing sites, modifications of nucleotide sequences encoding a protein of interest, amino acid and/or protein fusions, and gene silencing by expressing an inverted repeat into a gene of interest.

### Transformation of Cells and Organisms

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

Transformation with a Recombinant Construct

The invention further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

Any polynucleotide encoding a Cas protein or other CRISPR system component disclosed herein may be functionally linked to a heterologous expression element, to facilitate transcription or regulation in a host cell. Such expression elements include but are not limited to: promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

### Introduction of a Cas Endonuclease Protein and a Guide RNA Polyribonucleotide

In some aspects, the Cas endonuclease and guide RNA may be introduced into the cell as a protein and a ribonuclease individually, or together as a ribonucleoprotein complex.

Following characterization of the guide RNA (or guide polynucleotide) and PAM sequence, a ribonucleoprotein (RNP) complex comprising the Cas endonuclease and the guide RNA (or guide polynucleotide) may be utilized to modify a target polynucleotide, including but not limited to: synthetic DNA, isolated genomic DNA, or chromosomal DNA in other organisms, including plants. To facilitate optimal expression and nuclear localization (for eukaryotic cells), the gene comprising the Cas endonculease may be optimized as described in WO2016186953 published 24 Nov. 2016, and then delivered into cells as DNA expression cassettes by methods known in the art. The components necessary to comprise an active RNP may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the complex may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof.

### Modification of Cells

As described herein, a guided Cas endonuclease can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9).

Alteration of the genome of a prokaryotic and eukaryotic cell or organism cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99).

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof. Protocols for introducing polynucleotides, polypeptides or polynucleotide-protein complexes into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN: 69PQBP; ISBN: 978-953-307-201-2), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, New York), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

In one aspect, the guide polynucleotide and/or Cas endonuclease are provided to the cell or target organism as a polynucleotide on a recombinant vector.

In one aspect, the guide polynucleotide/Cas endonuclease complex is a complex wherein the guide RNA and Cas endonuclease protein forming the guide RNA/Cas endonuclease complex are introduced into the cell as RNA and protein, respectively.

In one aspect, the guide polynucleotide/Cas endonuclease complex is a complex wherein the guide RNA and Cas endonuclease protein and the at least one protein subunit of a Cas protein forming the guide RNA/Cas endonuclease complex are introduced into the cell as RNA and proteins, respectively.

In one aspect, the guide polynucleotide/Cas endonuclease complex is a complex wherein the guide RNA and Cas endonuclease protein and the at least one protein subunit of a Cascade forming the guide RNA/Cas endonuclease complex (cleavage ready cascade) are preassembled in vitro and introduced into the cell as a ribonucleotide-protein complex.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Gene Targeting

The guide polynucleotide/Cas systems described herein can be used for gene targeting.

In general, DNA targeting can be performed by cleaving one or both strands at a specific polynucleotide sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends” or “staggered end”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

In one embodiment, the invention describes a method for modifying a target site in the genome of a cell, the method comprising introducing into a cell at least one Cas endonuclease and one guide RNA, and identifying at least one cell that has a modification at said target, wherein the modification at said target site is selected from the group consisting of insertion of at least one nucleotide, deletion of at least one nucleotide, replacement or substitution of at least one nucleotide, chemical modification of at least one nucleotide, or any combination of the preceding.

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J. 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

### Cells and Plants

The presently disclosed polynucleotides and polypeptides can be introduced into a plant cell. Any plant can be used with the compositions and methods described herein, including monocot and dicot plants, and plant elements.

In one aspect, it may be desirable to delete one or more nucleotides. In another aspect, it may be desirable to insert one or more nucleotides. In one aspect, it may be desirable to replace one or more nucleotides. In another aspect, it may be desirable to modify one or more nucleotides via a covalent or non-covalent interaction with another atom or molecule. In some aspects, the cell is diploid. In some aspects, the cell is haploid.

Genome modification via a Cas endonuclease-guide RNA complex may be used to effect a genotypic and/or phenotypic change on the target organism. Such a change is preferably related to an improved trait of interest or an agronomically-important characteristic, the correction of an endogenous defect, or the expression of some type of expression marker. In some aspects, the trait of interest or agronomically-important characteristic is related to the overall health, fitness, or fertility of the plant, the yield of a plant product, the ecological fitness of the plant, or the environmental stability of the plant. In some aspects, the trait of interest or agronomically-important characteristic is selected from the group consisting of: agronomics, herbicide resistance, insecticide resistance, disease resistance, nematode resistance, microbial resistance, fungal resistance, viral resistance, fertility or sterility, grain characteristics, commercial product production. In some aspects, the trait of interest or agronomically-important characteristic is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered starch content, altered carbohydrate content, altered sugar content, altered fiber content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

The meaning of abbreviations is as follows: “sec” means second(s), “min” means minute(s), “h” means hour(s), “d” means day(s), “μL” or “uL” or “ul” means microliter(s), “mL” means milliliter(s), “L” means liter(s), “μM” means micromolar, “mM” means millimolar, “M” means molar, “mmol” means millimole(s), “μmole” or “μmole” mean micromole(s), “g” means gram(s), “μg” or “ug” means microgram(s), “ng” means nanogram(s), “U” means unit(s), “bp” means base pair(s) and “kb” means kilobase(s).

Knockouts of the Zea mays NAC7 transcription factor with an RNAi construct has been demonstrated to result in improved yield of maize plants under optimal growth conditions by delaying leaf senescence. However, some events also exhibited knock-down expression of other NAC family members, as well as increase in grain moisture. To specifically knockout ZM-NAC7 and assess yield advantage, we generated single gene knockout (KO) plants in Variety A using CRISPR/Cas9 editing tool, and studied their phenotypes. Specific deletion of ZM-NAC7 by CRISPR/Cas9 can delay leaf senescence that may lead to increase photosynthesis period and improve source capacity. Kernel number and kernel weight per ear at maturity were increased based on measurements for ear traits. No significant increase in grain moisture after partial deletion of NAC7 was observed.

### Example 1: Generation of ZM-NAC7 Mutant Alleles by Genome Editing

The genomic polynucleotide sequence of the Zea mays NAC7 gene in maize Variety A is given as SEQID NO:1, with the CDS given as SEQID NO:2 and the amino acid sequence given as SEQID NO:3.

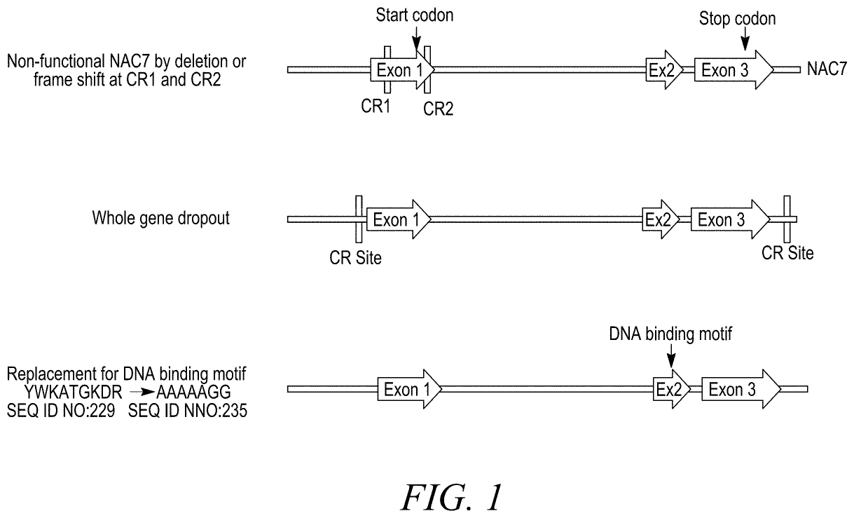

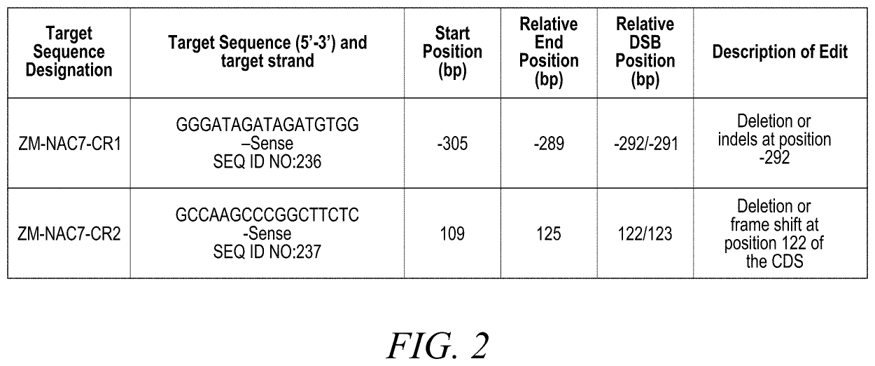

To generate knockout variants of ZM-NAC7, two guide RNAs at CR1 and CR2 sites were designed (Table 2) for target sequences SEQID NO:236 (CR1) and SEQID NO:237 (CR2). Guide RNA gRNA1 (SEQID NO:14) followed by AGG was designed to cut at the position between −292 and −291 upstream of the start codon ATG (FIG. 1). Guide RNA gRNA2 (SEQID NO:15) followed by CGG was designed to cut at the position between 122 and 123 (numbered from the start codon ATG). Therefore, perfect cleavages on both CR1 and CR2 would lead to a 413 bp deletion as described in FIG. 2.

The two gRNAs were built into a single plasmid construct in two expression cassettes both under the controls of ZM-U6 POLIII CHR8 promoter (SEQID NO:35) and ZM-U6 POLIII CHR8 terminator (TTTTTTTT). Via point particle-gun bombardment, the efficiency of these two gRNAs were tested in a transient embryo assay. CR1 showed transformation efficiency of 2.11% and CR2 of 1.1%. In addition, large fragment deletions between the two cleavage sites were identified.

Next, a plasmid was transformed through bombardment into immature maize embryos together with a helper plasmid that carried coding cassettes for the Cas9 protein (SEQID NO:30 encoded by SEQID NO:37), color marker AmCYAN1 (SEQID NO:31), and NPTII resistance (SEQID NO:32), the 2^{nd} helper plasmid with ZmODP2 (SEQID NO:33) and Kanamycin resistance (SEQID NO:34), and a plasmid containing coding cassettes for ZmWUS2 (SEQID NO:36) and Kanamycin resistance (SEQID NO:34). Selected plantlets from embryo callus were transferred to soil and allowed to grow into full plants. In total, 27 TO plants were identified and analyzed.

### Example 2: Identification of ZM-NAC7 Mutants from Edited Events

At V3-V4 stage, all TO plants were sampled for genotyping with two sets of PCR primers and three PCR reactions to amplify the surrounding regions of CR1 and CR2 individually and the combined CR1-CR2 region. Primers designed were: GSP1 (SEQID NO:10), GSP2 (SEQID NO:11), GSP3 (SEQID NO:12), and GSP4 (SEQID NO:13). PCR reactions on wildtype plants yielded products of 78 bp, 104 bp, and 511 bp. The three different PCR products from each TO plant were sequenced by NextGen Illumina sequencing. The sequence of Event 1 amplified by GSP1 and GSP2 is given as SEQID NO:4. The sequence of Event 1 amplified by GSP3 and GSP4 is given as SEQID NO:5. The sequence of Event 2 amplified by GSP1 and GSP2 is given as SEQID NO:6. The sequence of Event 2 amplified by GSP3 and GSP4 is given as SEQID NO:7. The sequence of Event 3 amplified by GSP1 and GSP2 is given as SEQID NO:8. The sequence of Event 3 amplified by GSP3 and GSP4 is given as SEQID NO:9.

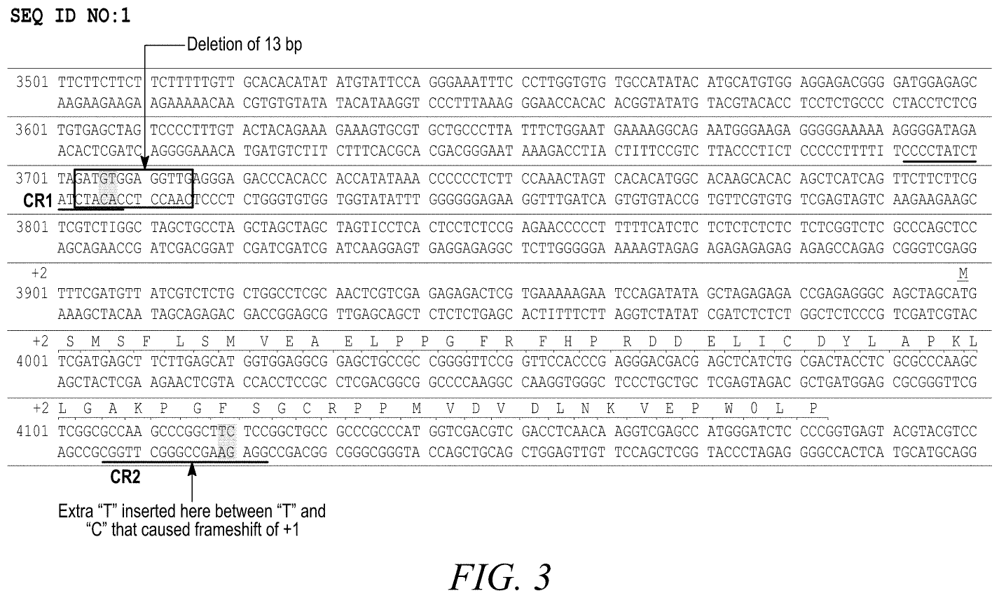

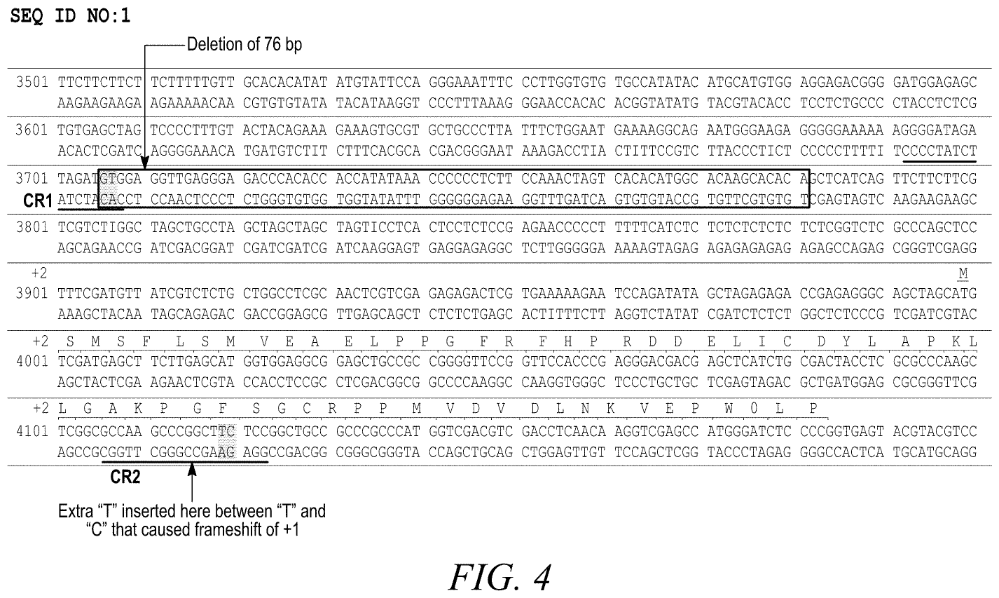

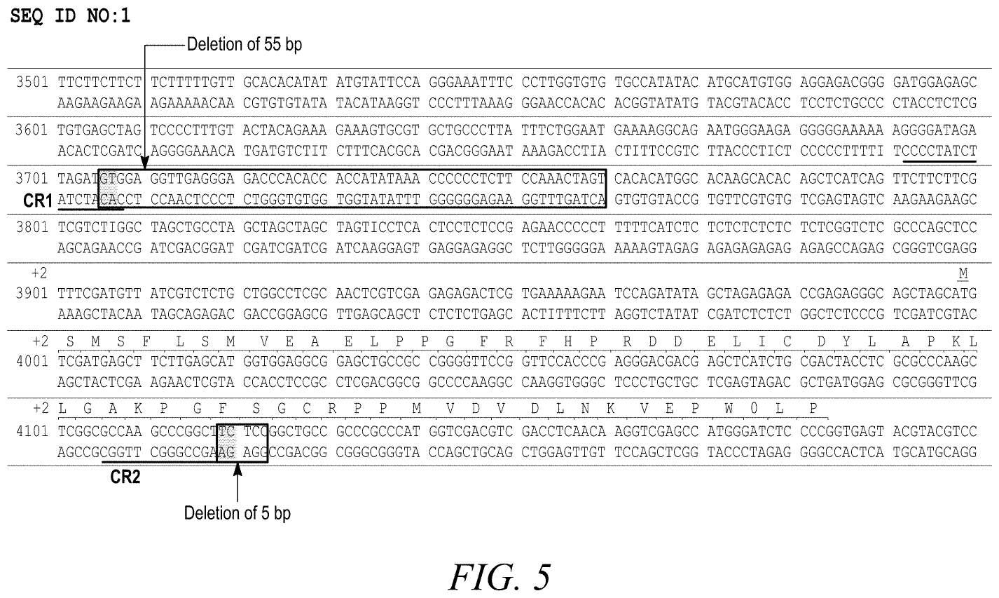

Out of the 27 TO plants, 15 were selected to move forward after genotyping. Seven of the 15 set seeds after being backcrossed to the parental maize event. Five of them were selected for T1 testing because their editing may have disrupted the ZM-NAC7 open reading frame, with one event later discarded because of vector backbone insertion. After genomic sequencing and vector plasmid detection analysis, T1 plants with the desired editing but without vector backbone insert for each TO parent were selected. As summarized in Table 3, Events 1, 2, and 3 had partial promoter deletion (−13 to 76 bp), and deletion (−5 bp) or insertion (+1 bp) at exon 1. The deletion or insertion at exon 1 is close to its N-terminus and before DNA binding motif of NAC7, demonstrating that the editing strategy created a non-functional NAC7 allele in all three events. The detailed sequence changes observed from Events 1, 2 and 3 are described in FIGS. 3, 4, and 5. These plants were backcrossed to the parental event again to generated heterozygous T2 seeds for field test, and also self-pollinated to produce segregating T2 seeds for phenotypic and grain yield studies.

### Example 3: ZM-NAC7 Knockout Edits Showed a Significant Delay in Senescence

### Dark-Induced Senescence

Dark-induced leaf senescence is a quick way to evaluate specific gene function in leaf senescence and stay-green. The experiment was done by detaching leaves and placing them in the dark. To examine the phenotype of ZM-NAC7 knockout edits, 7 inches of V15 leaf tips were collected and put into 50 mL Falcon tubes with 10 mL of deionized water. The tubes were placed in an incubator at 22° C. with 78% relative humidity in the constant dark.

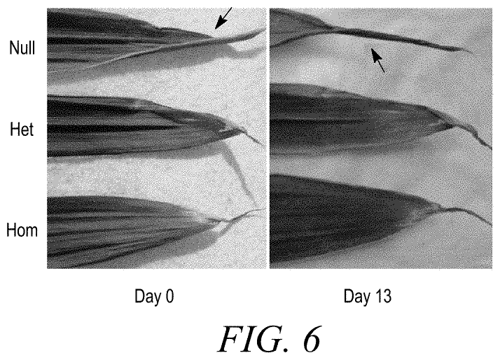

By day 10, a noticeable difference in leaf color between the Null leaves and the heterozygous (Het) and homozygous (Hom) leaves were clearly observed. Leaf tips from the Hom and Het knockout (KO) plants of all three events demonstrated delays in leaf senescence when compared with Nulls at day 13 of dark treatment (FIG. 6). Pictures shown in FIG. 6 were taken at days 0 and 13. Results are representatives of two independent experiments performed in triplicates. ZM-NAC7 knockout edits demonstrated a stay-green phenotype.

### Quantification of Chlorophyll Level in ZM-NAC7 KO Edits

In the dark-induced senescence experiment, the Het leaves showed an intermediate stay-green phenotype between Null and Hom leaves. To address whether the stay-green phenotype was dependent on the expression level of ZM-NAC7, the chlorophyll content in dark treated leaves of three genotypes were compared. Dualex instrument was used to measure chlorophyll level at 11 am for 12 days. Tables 4a and 4b show before dark treatment (day 0), there wasn't significant difference in chlorophyll level among Null, Het and Hom leaves, except for Event 3. However, at day 12, at construct level, both Het and Horn leaves had a significant higher chlorophyll content than those of Null leaves (26.06 μg/cm²). The chlorophyll content of Het and Horn leaves was similar (35.02 and 38.07 μg/cm², respectively). The stay-green effect caused by knockout of ZM-NAC7 may be dominant in Het plants.

### Example 4: Deletion of ZM-NAC7 Increased Kernel Number and Kernel Weight Per Ear

### Kernel Number Increases in ZM-NAC7 Knockout Edits

To evaluate potential yield efficacy of ZM-NAC7 knockout edits, plants were grown to maturity in the greenhouse. After a black layer appeared, ears were harvested. Ear photometry images were taken from each plant and analyzed for ear component traits. After that, ears were shelled by hand and kernel number per ear were counted by a DRELLO seed counter.

Tables 5a and 5b show that both Event 2 and 3 had kernel number increase compared with Null. Between these two, Event 2 had the higher yield efficacy potentials. Kernel number of Horn plants of Event 2 was significantly higher than that of Null plants (p=0.0043). At the construct level, average kernel number per ear of Null, Het and Horn is 241, 264 and 296 kernels, respectively. There is significantly statistical difference in kernel increase between Horn and Null (p=0.0378) in construct level. Horn plants of Event 1 didn't show kernel number increase, although it showed stay-green phenotype as listed in Tables 4a and 4b.

### Kernel Weight Increases in ZM-NAC7 Knockout Edits

In addition to kernel number increase, we also observed kernel weight increase from ZM-NAC7 knockout plants. Shelled kernels were oven dried at 70° C. for 72 hr after harvest. Tables 6a and 6b demonstrate that, at the construct level, average kernel dry weight per ear showed the similar upward trend from null (50.93 g), to Het (55.53 g) and Horn (63.15 g). The kernel weight increase from Null to Horn is statistically different (p=0.0273) at construct level.

There was no significant increase in single kernel weight among Null, Het and Horn plants (data not shown). Our findings demonstrated that partial deletion of ZM-NAC led to yield per plant increase, caused by kernel number increase and kernel weight per ear increase.

### Example 5: Ear Components of ZM-NAC7 Edits Studied by Ear Photometry

To identify key ear traits that determine yield increase in ZM-NAC7 knockout edits, ear component parameters were measured by ear photometry image analysis. Partial deletion of ZM-NAC7 increased kernel number (Tables 7a and 7b) and ear volume (Tables 8a and 8b) based on photometry data. Similar to the results obtained from the kernel counter, Event 2 was more efficacious in kernel number increase as Hom plants have average of 354 kernels/ear compared with 236 kernels/ear from Null plants (p=0.0065). Overall at the construct level, both kernel number and ear volume showed significantly increase from Null to Hom with p=0.0421 and p=0.034, respectively. The improvement on these beneficial ear traits are attributes for yield increase caused by ZM-NAC7 knockout.

### Example 6: Partial Deletion of NAC7 Didn't Increase Kernel Moisture

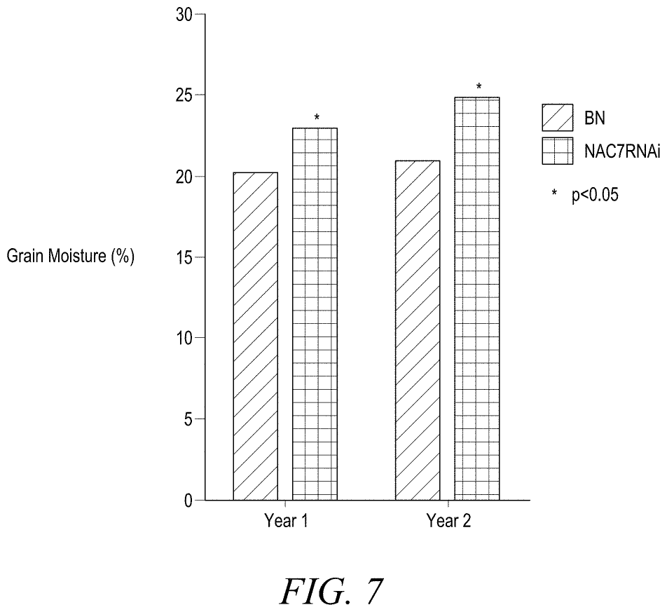

To evaluate if deletion of NAC7 and delayed senescence caused grain moisture increase, kernel moisture of NAC7 KO edits was determined by the drying method using methodologies standard in the art (see, for example, Risius et al., Biosystems Engineering 156:120-135, 2017; and Hurburgh et al., Transactions of the ASAE 28:634-640, 1985), shown as the percentage of (fresh weight−dry weight)/fresh weight in Tables 9a and 9b. Kernel fresh weight was measured right after the harvest. Dry weight (Table 5b) was measured for each cob after kernels were removed from the cob, and were oven dried at 70° C. for 72 hr. At the construct level, average grain moisture of Null, Het and Hom plants was 19.7%, 20.95% and 20.93%, respectively. There was no statistical difference between Null, Het and Hom in all 3 events. FIG. 7 shows harvest moisture of hybrids that expressing UBI:NAC7 RNAi, as a comparison. Harvest moisture of hybrids expressing THE NAC7 RNAI CONSTRUCT (UBI:NAC7 RNAi) is presented. Statistical difference between THE NAC7 RNAI CONSTRUCT and the bulk null at construct level were determined by the mixed model with spatial adjustment (Gilmour et al., 2009). In two years' field test, plants with NAC7 RNAi transgene had 2-4% grain moisture increase. Our data demonstrated that CRISPR-Cas edited NAC7 KO plants with specific NAC7 deletion minimized grain moisture increase while providing improved yield.

### Example 7: Hybrid Yield Test for NAC7 KO Edits

As described above, we observed that both Hom and Het plants with ZM-NAC7 deletion showed stay-green phenotype and increases in ear component parameters, such as kernel number per ear, kernel weight per ear, and ear volume. Although Hom plants were more efficacious for kernel number increase compared with Het plants, there is no statistical difference in chlorophyll content between Hom and Het plants. Yield improvements may be obtained in maize with endogenous NAC7 gene edits in only one allele.

### Example 8: Additional Strategies to Generate NAC7 KO Edits and Downregulated NAC7 Function

In addition to the ZM-NAC7 partial deletion in Variety A as discussed above, three additional editing protocols for ZM-NAC7 are tested.

### Whole Gene Deletion

Two guide RNAs are used to generate full gene dropout in Variety B and Variety C as illustrated in the middle panel of FIG. 1. These two edits from both NSS and SS will enable testing of the hybrid yield efficacy of ZM-NAC7 deletion, with a potential stronger efficacy as demonstrated from Hom inbred plants as described above. Guide RNA sequences for the gene deletion experiments are given as SEQID NOs:16-19.

### Modulate Promoter Region of NAC7 by Genome Editing

Cis-regulatory elements in promoter region may regulate ZM-NAC7 expression and function. Therefore, an alternative approach to downregulate expression of NAC7 is achieved by deleting selected nucleotides in the upstream expression element promoter region. In Variety A, HC69 and Variety B, around 1.2 Kb sequence upstream of the ATG is highly conserved. There is also a significant amount of repetitive DNA present in the promoter region of NAC7. These sequences are targeted for editing to modulate NAC7 expression. Guide RNA sequences for the promoter editing experiment are given as SEQID NOs:22-29.

### Example 9: Structural Analysis and DNA Binding Motif Modification of NAC Proteins

There are two NAC7 homologs with 3D structure solved (Arabidopsis NAC domain-containing protein 19, SEQID NO:227 and Oryza sativa PDB:3ULX, SEQID NO:228). Both structures exhibited similarity, with the Arabidopsis variant additionally having an oligo-DNA bound. Based on the Arabidopsis structure (PDB:3 SWM, Weiner et al, Biochem 7 444:395-404, 2012), a ZmNAC7 model was built.

### N-Terminal Region

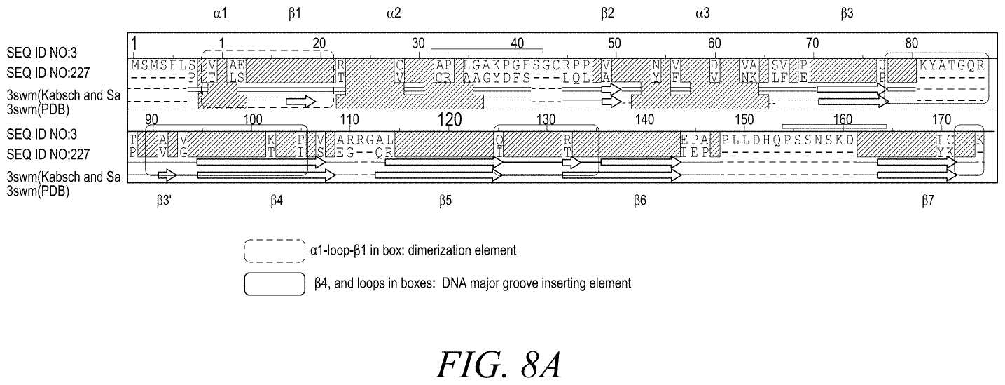

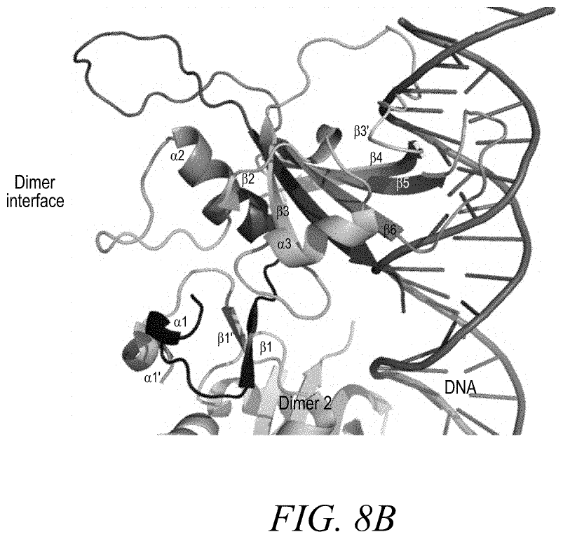

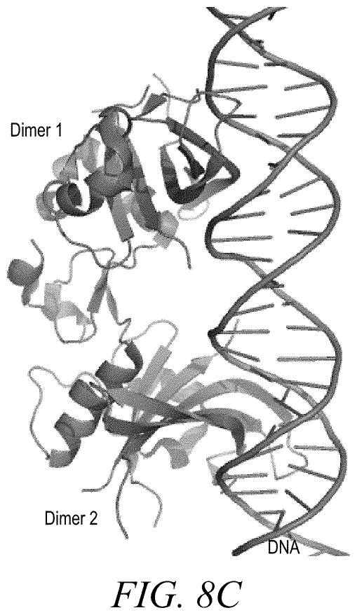

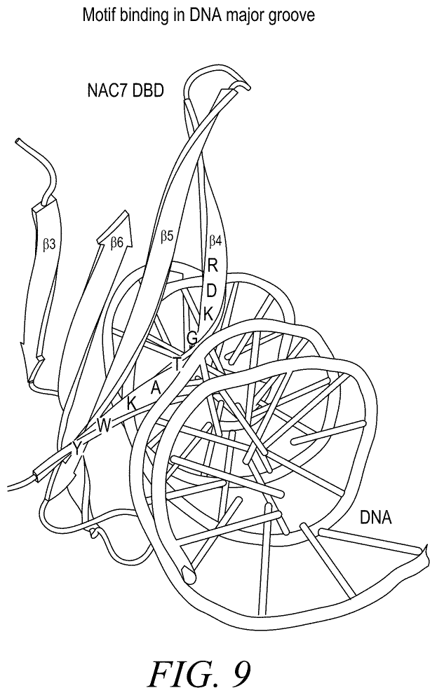

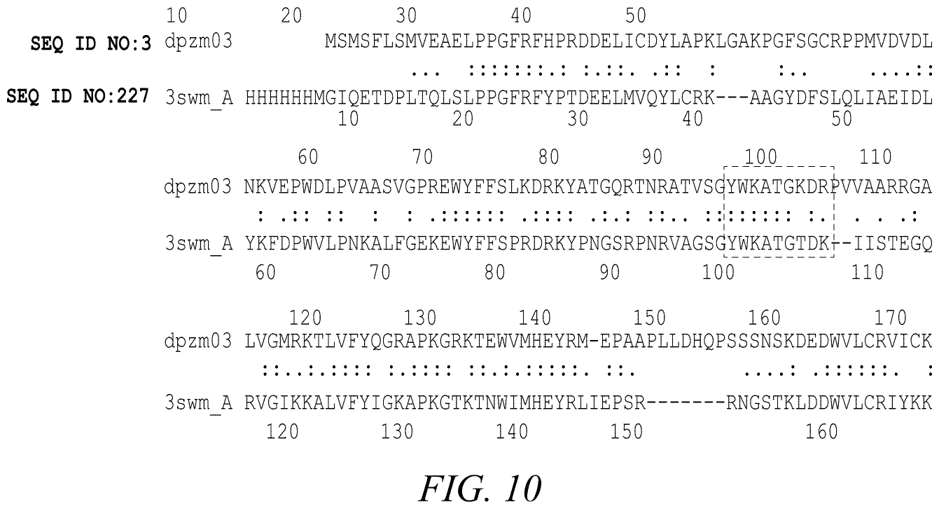

NAC7's N-terminal domain (1-174 aa) binds the DNA duplex. The major structure core consisted of a 6 stranded beta sheet (β2-β3-β7-β6-β5-β4) (FIG. 8A). The alpha helices α2 and α3 flanked the sheet's β2-β3 on both sides while the β5-β4 curled significantly forming semi-barrel with help of β3′ (FIG. 8B). NAC7 functions as a homodimer (FIG. 8C). The dimer is related with 2-fold axis and its interface is formed by N-terminal peptide including α1-loop-β1. The central β-sheet's β4 edge (with the motif YWKATGKDR (SEQID NO:229)) inserts into the DNA duplex major groove, determining the sequence recognition specificity (FIG. 9). This motif in the Zea mays NAC7 variant sequence (SEQID NO:3) is shown as a dashed line box on FIG. 10. Other elements including loops spanning β3-β3′ loop, β5-β6, and β7-C-ter interact with the DNA phosphate backbone, mainly providing binding energy.

### C-Terminal Region

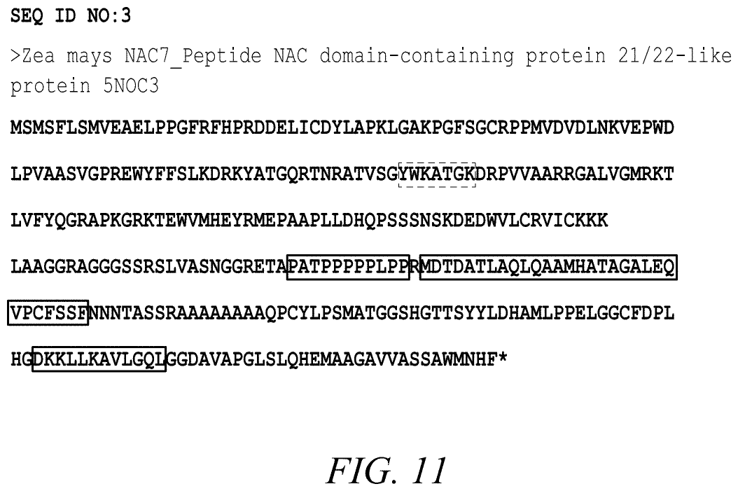

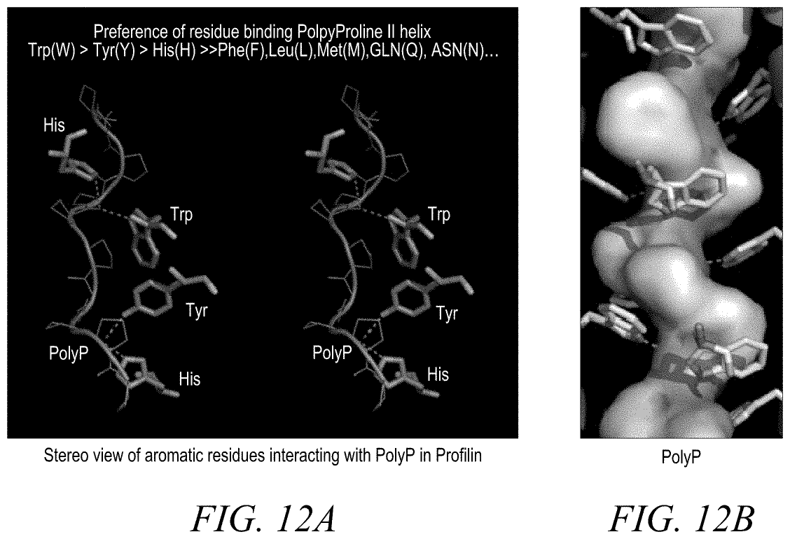

The C-terminal region had relatively low sequence complexity, with mainly hydrophilic residues. It belongs to a so-called intrinsic disordered protein, suitable for protein-protein interaction. Related proteins from uniref90_plant database with homology on the C-terminal domain were aligned together. Conserved regions of the Zea mays NAC7 variant (SEQID NO:3), consistent with the predicted helix areas, are shown as solid line boxed areas in FIG. 11. The polyproline segment (PATPPPPPLPP (SEQID NO:230)) is associated with protein recognition, and usually assumes polyproline II helix structure, providing an excellent docking site for aromatic residues (FIGS. 12A and 12B). There was also an important C-terminal motif (AAGAVVASSAWMNHF (SEQID NO:231)).

### Modify DNA Binding Motif of ZM-NAC7 by Fragment Replacement

Protein structural model of NAC7 shows the central β-sheet (amino acid sequence: YWKATGKDR given as SEQID NO:229) inserts into DNA duplex major groove, which likely determines the recognition specificity of NAC7 transcription factor activity. Other elements, including loops spanning β3-β3′ loop, β5-β6, and β7-C-ter, interact with the DNA phosphate backbone and provide binding energy. To downregulate the function of Zm-NAC7 without interrupting the activity of its potential interactors, an NAC7 edit with modified DNA binding motif to abolish its DNA binding capability was tested (lower panel of FIG. 1). The amino acids “AAAAAGG” (SEQID NO:235) substituted the DNA binding motif “YWKATGK” (SEQID NO:229) in ZM-NAC7 by guided Cas9, as shown in FIG. 1. Guide RNA sequences for the binding motif null are given as SEQID NOs:20-21.

FIG. 9 shows the motif of the protein binding region in the DNA major groove. NAC7 comprises two major domains, the N-terminal (1-174 aa) DNA binding domain (DBD) and the C-terminal (175-338 aa) intrinsic disorder domain (ID).

### Example 10: Sequence Analysis of NAC Proteins

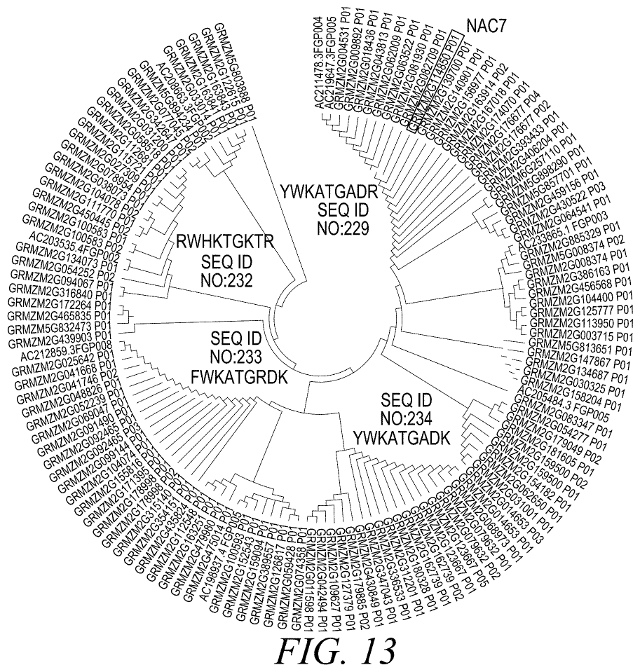

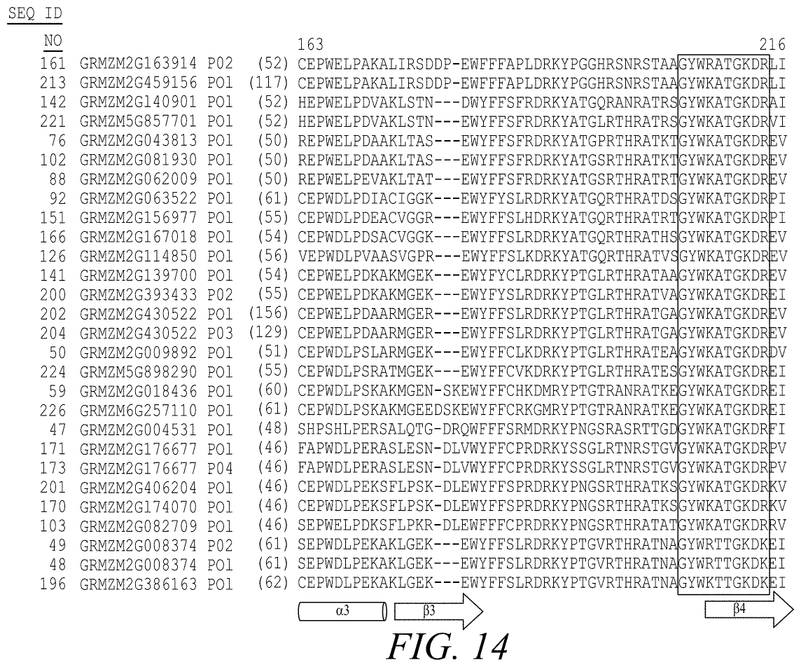

Over 150 Maize NAC sequences (SEQID NOs:38-226) were identified and analyzed, and a phylogenetic tree created based on the sequence motif implicated in target DNA binding, the central β-sheet's β4 edge region. Table 1 lists some of the key motifs for NAC7 protein activity. Any variation in any of the sequences or motifs described herein has the potential to alter the specificity and/or affinity of the NAC protein. FIG. 13 shows the phylogenetic tree for the maize NAC proteins, with clustering of the sequences for the central β-sheet's β4 edge region, where it inserts into the DNA duplex major groove (as described above). Table 10 lists some of the motif variations in maize for the sequences corresponding to the β-sheet's β4 edge region. FIG. 14 shows a sequence alignment, with the β-sheet's β4 edge region variations outlined in black boxes. Table 11 shows the frequency of occurrence of different conserved motifs.

The gene editing methods described herein may be used to modify any NAC gene in any plant, including crop plants, such as but not limited to maize, soybean, cotton, canola, wheat, sorghum, sunflower, barley, or rice, to effect modulation or knockout of expression or activity, to improve a trait of agronomic or commercial importance.

### Tables

TABLE 1
Sequence Motifs of NAC proteins

| Description | Sequence |
|---|---|
| central β-sheet's β4 edge | YWKATGKDR |
| motif consensus variation 1 | |
| central β-sheet's β4 edge | RWHKTGKTR |
| motif consensus variation 2 | |
| central β-sheet's β4 edge | FWKATGRDK |
| motif consensus variation 3 | |
| central β-sheet's β4 edge | YWKATGADK |
| motif consensus variation 4 | |
| polyproline segment | PATPPPPPLPP |
| associated with protein | |
| recognition | |
| C-terminal motif | AAGAVVASSAWMNHF |

TABLE 2
Experimental rationale

| | Description of | |
|---|---|---|
| Editing rationale | genome edits | Target Position |
| Delete part of the promoter | In frame deletion or | −291 to 122 in NAC7 |
| and part of the first exon/ | out-of-frame edit | Variety A gene |
| coding region | | |

TABLE 3
Edited plants show partial promoter deletion,
and exon 1 deletion or an insertion

| Event | Modification at CR sites |
|---|---|
| Event 1 | CR1: −13bp; CR2: +1bp |
| Event 2 | CR1: −55bp; CR2: −5bp |
| Event 3 | CR1: −76bp; CR2: +1bp |

TABLE 4a
Knock out of NAC7 delayed senescence and increased
chlorophyll level (ug/cm²) in leaf

| | | Day 0 | | | Day 12 | | |
|---|---|---|---|---|---|---|---|
| | | Least | | Signi- | Least | | Signi- |
| | Geno- | Sq | Std | ficance | Sq | Std | ficance |
| | type | Mean | Error | level | Mean | Error | level |
| Event 1 | Null | 43.53 | 0.99 | A | 28.05 | 2.01 | A |
| | Het | 46.48 | 0.99 | A | 34.50 | 2.01 | A |
| | Hom | 46.15 | 0.99 | A | 41.81 | 2.01 | B |
| Event 2 | Null | 43.04 | 2.11 | A | 20.28 | 2.36 | A |
| | Het | 43.04 | 2.11 | A | 37.01 | 2.36 | B |
| | Hom | 45.68 | 2.11 | A | 34.64 | 2.36 | B |
| Event 3 | Null | 46.00 | 1.92 | A | 29.86 | 2.07 | A |
| | Het | 42.74 | 1.92 | AB | 33.56 | 2.07 | AB |
| | Hom | 36.91 | 1.92 | B | 37.76 | 2.07 | B |
| Aggregate | Null | 44.19 | 1.11 | A | 26.06 | 1.34 | A |
| | Het | 44.09 | 1.11 | A | 35.02 | 1.34 | B |
| | Hom | 42.91 | 1.11 | A | 38.07 | 1.34 | B |

TABLE 4b
Knock out of NAC7 delayed senescence and increased
chlorophyll level (ug/cm²) in leaf

| | Geno- | Geno- | Day 0 | | Day 12 | |
|---|---|---|---|---|---|---|
| | type | type | Difference | p-Value | Difference | p-Value |
| Event 1 | Hom | Null | 2.62 | 0.1662 | 13.76 | 0.0002 |
| | Hom | Het | −0.33 | 0.9701 | 7.31 | 0.0428 |
| | Het | Null | 2.95 | 0.1079 | 6.45 | 0.0801 |
| Event 2 | Hom | Null | 2.64 | 0.6537 | 14.35 | 0.0007 |
| | Hom | Het | 2.64 | 0.6538 | −2.37 | 0.7591 |
| | Het | Null | 0.00 | 1.0000 | 16.72 | 0.0001 |
| Event 3 | Hom | Null | −9.08 | 0.0074 | 7.91 | 0.0321 |
| | Hom | Het | −5.83 | 0.1019 | 4.20 | 0.3389 |
| | Het | Null | −3.25 | 0.4671 | 3.71 | 0.4272 |
| Aggregate | Hom | Null | −1.27 | 0.6955 | 12.01 | <.0001 |
| | Hom | Het | −1.17 | 0.7345 | 3.05 | 0.246 |
| | Het | Null | −0.10 | 0.9977 | 8.96 | <.0001 |

TABLE 5a
Knock out of NAC7 increased kernel number per ear

| | | Least Sq | Std | Significance |
|---|---|---|---|---|
| | Genotype | Mean | Error | level |
| Event 1 | Null | 279.93 | 27.65 | A |
| | Het | 254.08 | 21.42 | A |
| | Hom | 241.09 | 32.29 | A |
| Event 2 | Null | 222.44 | 23.69 | A |
| | Het | 266.30 | 21.19 | AB |
| | Hom | 334.81 | 23.69 | B |
| Event 3 | Null | 219.93 | 31.06 | A |
| | Het | 273.53 | 26.66 | A |
| | Hom | 295.85 | 25.99 | A |
| Aggregate | Null | 240.82 | 16.00 | A |
| | Het | 263.67 | 13.41 | AB |
| | Hom | 296.30 | 15.65 | B |

TABLE 5b
Knock out of NAC7 increased kernel number per ear

| | Genotype | Genotype | Difference | p-Value |
|---|---|---|---|---|
| Event 1 | Hom | Null | −38.84 | 0.6344 |
| | Hom | Het | −12.99 | 0.9400 |
| | Het | Null | −25.85 | 0.7416 |
| Event 2 | Hom | Null | 112.38 | 0.0043 |
| | Hom | Het | 68.51 | 0.0892 |
| | Het | Null | 43.86 | 0.3591 |
| Event 3 | Hom | Null | 75.92 | 0.1567 |
| | Hom | Het | 22.32 | 0.3968 |
| | Het | Null | 53.60 | 0.8210 |
| Aggregate | Hom | Null | 55.48 | 0.0378 |
| | Hom | Het | 32.63 | 0.2562 |
| | Het | Null | 22.85 | 0.5189 |

TABLE 6a
Knock out of NAC7 increased kernel dry weight (g) per ear

| | | Least Sq | Std | Significance |
|---|---|---|---|---|
| | Genotype | Mean | Error | level |
| Event 1 | Null | 60.80 | 5.97 | A |
| | Het | 54.41 | 4.63 | A |
| | Hom | 55.04 | 6.98 | A |
| Event 2 | Null | 43.31 | 5.32 | A |
| | Het | 54.29 | 4.76 | A |
| | Hom | 59.89 | 5.01 | A |
| Event 3 | Null | 49.07 | 5.97 | A |
| | Het | 58.31 | 5.13 | AB |
| | Hom | 71.37 | 5.27 | B |
| Aggregate | Null | 50.93 | 3.36 | A |
| | Het | 55.53 | 2.81 | AB |
| | Hom | 63.15 | 3.28 | B |

TABLE 6b
Knock out of NAC7 increased kernel dry weight (g) per ear

| | Genotype | Genotype | Difference | p-Value |
|---|---|---|---|---|
| Event 1 | Hom | Null | −5.76 | 0.81 |
| | Hom | Het | 0.63 | 1.00 |
| | Het | Null | −6.40 | 0.68 |
| Event 2 | Hom | Null | 16.58 | 0.0694 |
| | Hom | Het | 5.61 | 0.6979 |
| | Het | Null | 10.98 | 0.2816 |
| Event 3 | Hom | Null | 22.30 | 0.0197 |
| | Hom | Het | 13.06 | 0.1881 |
| | Het | Null | 9.23 | 0.4749 |
| Aggregate | Hom | Null | 12.22 | 0.0273 |
| | Hom | Het | 7.63 | 0.1854 |
| | Het | Null | 4.59 | 0.5472 |

TABLE 7a
Ear photometry result showed that knock out
of NAC7 increased kernel number per ear

| | | Least Sq | Std | Significance |
|---|---|---|---|---|
| | Genotype | Mean | Error | level |
| Event 1 | Null | 319.24 | 32.20 | A |
| | Het | 292.96 | 24.94 | A |
| | Hom | 286.06 | 37.60 | A |
| Event 2 | Null | 236.35 | 26.40 | A |
| | Het | 298.49 | 24.23 | AB |
| | Hom | 354.43 | 25.61 | B |
| Event 3 | Null | 262.72 | 35.10 | A |
| | Het | 322.32 | 30.13 | A |
| | Hom | 341.88 | 29.37 | A |
| Aggregate | Null | 272.18 | 18.09 | A |
| | Het | 303.48 | 15.29 | AB |
| | Hom | 333.53 | 17.52 | B |

TABLE 7b
Ear photometry result showed that knock out
of NAC7 increased kernel number per ear

| | Genotype | Genotype | Difference | p-Value |
|---|---|---|---|---|
| Event 1 | Hom | Null | −33.18 | 0.7817 |
| | Hom | Het | −6.90 | 0.9872 |
| | Het | Null | −26.29 | 0.7959 |
| Event 2 | Hom | Null | 118.08 | 0.0065 |
| | Hom | Het | 55.94 | 0.2610 |
| | Het | Null | 62.14 | 0.2029 |
| Event 3 | Hom | Null | 79.16 | 0.2043 |
| | Hom | Het | 19.56 | 0.8880 |
| | Het | Null | 59.60 | 0.4082 |
| Aggregate | Hom | Null | 61.35 | 0.0421 |
| | Hom | Het | 30.05 | 0.4017 |
| | Het | Null | 31.30 | 0.3856 |

TABLE 8a
Ear photometry result showed that knock
out of NAC7 increased ear volume (cm³)

| | | Least Sq | Std | Significance |
|---|---|---|---|---|
| | Genotype | Mean | Error | level |
| Event 1 | Null | 695.31 | 26.32 | A |
| | Het | 722.28 | 21.73 | A |
| | Hom | 681.54 | 30.73 | A |
| Event 2 | Null | 582.28 | 31.96 | A |
| | Het | 652.73 | 29.33 | A |
| | Hom | 680.11 | 30.13 | A |
| Event 3 | Null | 636.16 | 37.83 | A |
| | Het | 675.80 | 32.47 | A |
| | Hom | 738.68 | 32.47 | A |
| Aggregate | Null | 636.72 | 19.07 | A |
| | Het | 685.53 | 16.52 | AB |
| | Hom | 703.62 | 18.47 | B |

TABLE 8b
Ear photometry result showed that knock
out of NAC7 increased ear volume (cm³)

| | Genotype | Genotype | Difference | p-Value |
|---|---|---|---|---|
| Event 1 | Hom | Null | −13.78 | 0.9382 |
| | Hom | Het | −40.74 | 0.5299 |
| | Het | Null | 26.96 | 0.7111 |
| Event 2 | Hom | Null | 97.83 | 0.0763 |
| | Hom | Het | 27.38 | 0.7926 |
| | Het | Null | 70.45 | 0.2450 |
| Event 3 | Hom | Null | 102.52 | 0.1097 |
| | Hom | Het | 62.88 | 0.3647 |
| | Het | Null | 39.63 | 0.7078 |
| Aggregate | Hom | Null | 66.90 | 0.0340 |
| | Hom | Het | 18.08 | 0.7462 |
| | Het | Null | 48.81 | 0.1326 |

TABLE 9a
Partial deletion of NAC7 didn't significantly
increase kernel moisture (%)

| | | Least Sq | Std | Significance |
|---|---|---|---|---|
| | Genotype | Mean | Error | level |
| Event 1 | Null | 20.32% | 1.09% | A |
| | Het | 21.93% | 0.84% | A |
| | Hom | 18.67% | 1.33% | A |
| Event 2 | Null | 20.49% | 1.02% | A |
| | Het | 20.95% | 0.91% | A |
| | Hom | 22.11% | 0.96% | A |
| Event 3 | Null | 18.12% | 1.06% | A |
| | Het | 19.59% | 0.90% | A |
| | Hom | 20.99% | 0.86% | A |
| Aggregate | Null | 19.73% | 0.62% | A |
| | Het | 20.95% | 0.52% | A |
| | Hom | 20.93% | 0.59% | A |

TABLE 9b
Partial deletion of NAC7 didn't significantly
increase kernel moisture (%)

| | Genotype | Genotype | Difference | p-Value |
|---|---|---|---|---|
| Event 1 | Hom | Null | −1.65% | 0.6062 |
| | Hom | Het | −3.26% | 0.1072 |
| | Het | Null | 1.61% | 0.4752 |
| Event 2 | Hom | Null | 1.62% | 0.4842 |
| | Hom | Het | 1.16% | 0.6587 |
| | Het | Null | 0.46% | 0.9391 |
| Event 3 | Hom | Null | 2.87% | 0.1011 |
| | Hom | Het | 1.40% | 0.5053 |
| | Het | Null | 1.47% | 0.5486 |
| Aggregate | Hom | Null | 1.19% | 0.3479 |
| | Hom | Het | −0.03% | 0.9994 |
| | Het | Null | 1.22% | 0.2891 |

TABLE 10
Exemplary NAC protein motif variations in maize

| SEQID | |
|---|---|
| NO: | motif |
| 238 | RWHKTGASK |
| 239 | RWHKTGKTK |
| 240 | RWHKTGKTR |
| 241 | RWHKTGNTK |
| 242 | RWHKTGRSK |
| 243 | RWHKTGSSK |
| 244 | RWKPSGKEK |
| 245 | RWRPAGKEK |
| 246 | TWHSEAKPK |
| 247 | VWRPSGKET |
| 248 | YWKAAGTPG |
| 249 | YWKATGADK |
| 250 | YWKATGADR |
| 251 | YWKATGKDC |
| 252 | YWKATGKDK |
| 253 | YWKATGKDR |
| 254 | YWKATGKEE |
| 255 | YWKATGKEK |
| 256 | YWKATGPDR |
| 257 | YWKATGSPS |
| 258 | YWKATGTDK |
| 259 | YWKITGKDC |
| 260 | YWKSTGKDR |
| 261 | YWKTSGKDR |
| 262 | YWKTTGKDK |
| 263 | YWKTTGKDR |
| 264 | YWNPAGADE |
| 265 | YWSSVCADE |

TABLE 11
Frequency of conserved motifs

| Motif | Occurrence |
|---|---|
| YWKATGKDR | 27 |
| FWKATGRDK | 18 |
| YWKATGADK | 13 |
| YWKATGTDK | 6 |
| YWKATGKDK | 6 |
| YWKSTGKDR | 4 |
| YWKATGADR | 4 |
| RWHKTGKTR | 4 |
| FWKATGTDR | 4 |
| FWKATGSDR | 4 |
| YWKTTGKDK | 3 |
| YWKAAGTPG | 3 |
| FWKATGIDR | 3 |
| YWKTTGKDR | 2 |
| YWKTSGKDR | 2 |
| YWKATGKEK | 2 |
| VWRPSGKET | 2 |
| RWRPAGKEK | 2 |
| RWHKTGRSK | 2 |
| YWSSVCADE | 1 |
| YWNPAGADE | 1 |
| YWKITGKDC | 1 |
| YWKATGSPS | 1 |
| YWKATGPDR | 1 |
| YWKATGKEE | 1 |
| YWKATGKDC | 1 |
| TWHSEAKPK | 1 |
| RWKPSGKEK | 1 |
| RWHKTGSSK | 1 |
| RWHKTGNTK | 1 |
| RWHKTGKTK | 1 |
| RWHKTGASK | 1 |
| LWKATGRDK | 1 |
| FWKATGIDK | 1 |
| CWRSDSGVK | 1 |
| CWKKIGSPR | 1 |
| CWKKIGSHI | 1 |
| CWHSEAGAK | 1 |
| CWHNEAKAR | 1 |

We claim:

1. A method of modifying an endogenous genomic locus of a plant, wherein the locus comprises a polynucleotide encoding a NAC polypeptide, said method comprising:
a. providing to at least one cell of the plant a molecular modification agent, and
b. introducing one or more genetic modifications at the genomic locus that results in a reduced expression of the NAC polypeptide or reduced activity of the NAC polypeptide, as compared to a control plant not comprising the one or more genetic modifications;
wherein the one or more genetic modifications is selected from the group consisting of:
an insertion of at least one nucleotide, the deletion of at least one nucleotide, the substitution of at least one nucleotide, the molecular alteration of at least one nucleotide, and any combination of the preceding.

2. The method of claim 1, wherein the one or more genetic modifications a the genomic locus results in improved health, improved stay-green phenotype, delayed senescence, or higher yield of the plant that comprises the one or more genetic modifications at the genomic locus.

3. The method of claim 1, wherein insertion or deletion of at least one nucleotide in or near the NAC coding region effects a frameshift in the endogenous NAC gene.

4. The method of claim 1, wherein a regulatory expression element of the endogenous NAC gene is altered.

5. The method of claim 1, wherein at least 5 bases of the endogenous NAC gene are deleted.

6. The method of claim 1, wherein at least 1 base of the endogenous NAC gene is inserted.

7. The method of claim 1, wherein a functional motif of the endogenous NAC gene is edited or replaced.

8. The method of claim 7, wherein the functional motif is selected from the group consisting of:
a. a DNA interaction domain comprising at least two beta sheets and a sequence comprising a tryptophan, an acidic residue, and a basic residue;
b. a protein recognition domain comprising an alpha helix with a plurality of proline residues; and
c. a C-terminal domain comprising a tryptophan.

9. The method of claim 1, wherein a plurality of sites of the endogenous NAC gene are altered.

10. The method of claim 9, wherein at least one of the plurality of sites is upstream of the coding region.

11. The method of claim 1, wherein the modification agent is a Cas endonuclease.

12. The method of claim 11, further comprising a guide polynucleotide that is capable of hybridizing with a sequence at or near the endogenous genomic locus.

13. The method of claim 1, wherein the plant is maize.

14. The method of claim 13, wherein the one or more genetic modifications a the genomic locus results in greater average kernel number per ear or greater average kernel dry weight per ear of the maize plant.

15. The method of claim 1, wherein the NAC polypeptide is NAC7.

16. The method of claim 15, wherein the average grain moisture of the kernels from a cob of a plant produced by the method is not more than 2% higher than that of a null control.

17. The method of claim 15, wherein the NAC7 polypeptide comprises a sequence that shares at least 80% sequence identity with SEQID NO:3.

18. The method of claim 1, comprising introducing a first modification at a position between −291 and −292 bases upstream of the start codon of the NAC polypeptide, and introducing a second modification at a position between 122 and 123 bases downstream of the start codon of the NAC polypeptide.

19. A method of altering the binding specificity of a NAC protein in a plant, the method comprising introducing an edit to a sequence motif comprising: N1-N2-N3-N4-N5-N6-N7-N8-N9, wherein:
a. N1=F, R, T, V, or Y;
b. N2=W;
c. N3=H, K, R, N, or S;
d. N4=S, P, T, I A, or K;
e. N5=T, S, A, V, or E;
f. N6=G, A, or C;
g. N7=R, K, A, S, T, P, or N;
h. N8=D, S, T, E, or P; or
i. N9=K, E, C, G, T, or R.

20. The method of claim 19, comprising edits of at least two of the positions in the motif.
